(19) United States
(12) Patent Application Publication
(10) Pub. No.: US 2014/0010769 A1
(43) Pub. Date: Jan. 9, 2014

(54) COLOR CORRECTING COMPOSITIONS AND METHODS OF USE THEREOF

(71) Applicants: Joseph Lomakin, Cambridge, MA (US); Soo-Young Kang, Newton, MA (US); Betty Yu, Cambridge, MA (US); Morgan Pilkenton, Medford, MA (US)

(72) Inventors: Joseph Lomakin, Cambridge, MA (US); Soo-Young Kang, Newton, MA (US); Betty Yu, Cambridge, MA (US); Morgan Pilkenton, Medford, MA (US)

(21) Appl. No.: 13/865,431

(22) Filed: Apr. 18, 2013

Related U.S. Application Data
(60) Provisional application No. 61/635,188, filed on Apr. 18, 2012.

(51) Int. Cl. A61K 8/895; A61Q 17/04; A61Q 1/02

(52) U.S. Cl. CPC A61K 8/895; A61Q 1/02; A61Q 17/04
USPC 424/59; 424/63

(57) ABSTRACT

Compositions and methods of for changing the color of skin or body are described.

### RELATED APPLICATIONS

This application is a non-provisional application claiming priority to U.S. provisional application Ser. No. 61/635,188, filed Apr. 18, 2012. The entire contents of this application is hereby incorporated by reference.

### BACKGROUND OF THE INVENTION

Consumer care products offer benefits that include sun protection, color coverage, and delivery of skincare actives. In each of these cases, the substantivity or the durability of these products requires repeat application over the course of the day to enable persistence of the effect.

For the case of traditional color cosmetics where pigments are deposited on the skin to provide coverage of nonuniform skin tone, frequent ‘touch ups’ are required to maintain the coverage. Furthermore, traditional color cosmetics do not address skin imperfections such as wrinkles, scars, and other skin textural imperfections. In fact, the pigment dispersion precipitates out of the formulation and agglomerates over time, further highlighting skin roughness and wrinkles.

For skincare formulations, benefit inducing actives are delivered to the skin to provide long-term benefits. Because of the time required to achieve a demonstrable skin benefit, user compliance remains an obstacle to frequent, repeat product application.

With respect to traditional sun protective compositions, product efficacy is depends on product durability. Current commercial products, however, require frequent product application to achieve the desired sun protection factor.

Therefore, long-lasting, durable consumer care compositions that deliver benefits such as antiaging performance, sun protection, and color coverage remain an unmet need.

### SUMMARY OF THE INVENTION

The compositions, films and methods disclosed herein provide a durable, natural looking, non-invasive way of enhancing the appearance of skin and body while imparting additional benefits through incorporation of active ingredients in the benefit inducing layer. One example of such a benefit is uniform skin tone. The present teachings are based, at least in part, on the discovery that durable, natural looking, non-invasive compositions that are used in cosmetic applications for masking skin and body imperfections are also useful in changing the color of the body or skin.

In some embodiments, provided herein is a color corrective composition comprising:
a) a foundation component; and
b) a coloring component; and
c) a stabilizer component, wherein the foundation and stabilizer components stabilize the coloring component in the composition and enhance the longevity of the color.

In some embodiments, provided herein is a method for changing the color of skin, comprising applying to skin a composition comprising the color corrective composition as disclosed herein.

In some embodiments, provided herein is a method of changing the color of skin comprising the steps of:
a) applying a color corrective composition to the skin;
b) applying a reactive reinforcing component to color corrective composition; and
c) applying a cross-linking component to said reactive reinforcing component, wherein said cross-linking component facilitates in situ cross-linking of the reactive reinforcing component and the color corrective composition, thereby changing the color of the skin.

In some embodiments, provided herein is a color corrective film prepared by a process comprising the steps of:
a) applying a color corrective composition;
b) applying a reactive reinforcing component to skin; and
c) applying a cross-linking component to said reactive reinforcing component, wherein said cross-linking component facilitates in situ cross-linking of the reactive reinforcing component and the color corrective composition, such that a color corrective film is formed on skin.

In some embodiments, provided herein is a color corrective composition comprising:
a) a foundation component; and
b) a coloring component; and
c) a stabilizer component, wherein the foundation and stabilizer components stabilize the coloring component in the composition and enhance the longevity of the color when coated with a film.

In some embodiments, the color corrective composition further comprises a sun protection component, and the foundation and stabilizer components enhance the sun protection when coated with a film.

In some embodiments, the color corrective composition may be used in a method for changing the color of skin, comprising applying to skin a composition comprising the color corrective composition, thereby coloring skin.

In some embodiments, a kit for use in changing skin color is provided, the kit comprising the color corrective composition described herein and instructions for use.

### BRIEF DESCRIPTION OF THE DRAWINGS

FIG. 1 shows photographs of a subject's face before and after application of an exemplary composition of the present teachings, as described in the Example 3.

FIG. 2A is a graph showing the improved in vitro UVB protection of an exemplary composition of the present teachings.

FIG. 2B is a graph showing the improved in vivo UVB protection achieved by an exemplary composition of the present teachings.

FIG. 3A is a baseline photograph of a test subject.

FIG. 3B is a photograph of the test subject 3 hours post-application of an exemplary color corrective composition.

FIG. 3C is a photograph of the test subject after splashing her face with water.

### DETAILED DESCRIPTION OF THE INVENTION

The benefit-enhancing compositions, films and methods disclosed herein provide, prolong and/or enhance a skin benefit imparted by a skin benefit inducing layer applied to the skin.

In at least some embodiments, the skin benefit inducing layer comprises color corrective compositions that reduce the appearance of skin and or body imperfections, wounds or dermatological disorders. In particular, the compositions, films and methods disclosed herein are useful for addressing the visual and tactile properties of discolored skin imperfections such as dark circles, age spots, acne, superficial blood vessels, rosacea, scars. Additionally, the compositions, films and methods disclosed herein can improve the appearance and tactile properties of healthy skin. Over already available solutions for these conditions in the market, the compositions, films and methods disclosed herein provide numerous advantages, including a more natural skin finish, longer lasting color correction, and longer lasting UV protection. The uniformity of the color deposition and the shade deposited by the compositions, films and methods disclosed herein are adjustable to meet a broad range of skin types and personal aesthetics.

The compositions, films and methods disclosed herein provide long lasting benefits such as color change or modulation and broad spectrum sunscreen function. These benefits may be extended to include loading of other benefit-inducing ingredients such as pharmaceutical compounds, magnetic beads, quantum dots, and other particulates. The compositions and films disclosed herein can be adjusted to be applicable to a wide range of skin tones. Compared to traditional color cosmetics, the pigment dispersion uniformity is improved, leading to more natural and longer lasting coverage. Furthermore, when used in conjunction with a coating film, such as the films described in described in PCT/US2011/050003, published as WO2012/030984, the teachings of which are incorporated herein by reference, the color corrective composition has additional beneficial properties. Compared to traditional sunscreens, the substantivity is increased, thereby providing improved durability and coverage of the skin surface, enabling a once daily application with no need for reapplication.

The compositions and films have many beneficial uses, such as coverage of undereye dark circles; coverage of pigmentary imperfections and disorders; reduction of redness or cosmetic coverage of rosacea; boosting of sun protection factor; and augmenting skin tone.

In some embodiments, the benefit-inducing layer described herein may comprise a color corrective composition and one or more additional therapeutic agents. In alternative embodiments, the benefit-inducing layer may comprise one or more therapeutic agents without the color corrective composition.

In at least one embodiment, the benefit conferred by the benefit-inducing layer is enhanced or lengthened by the methods disclosed herein. For example, in certain embodiments, the activity of the therapeutic agent (e.g. antiaging/acne retinoids; eczema or psoriasis treatments; treatments for wound healing; antibacterials; and/or cosmetic ingredients) included in the benefit-inducing layer are enhanced or lengthened. Benefits include longer retention of the agent on the skin, less dilution of the agent; and/or enhanced visual effects once the agent is coated with the film.

In at least one embodiment, the present teachings relate to a matrix for providing attachment or entrapment of one or more therapeutic agents to the skin.

In some embodiments, the therapeutic agent may be a moisturizer, mineral oil, petroleum jelly, coal tar, anthralin, a corticosteroid, fluocinonide, vitamin D₃ analogues, retinoids, phototherapy, methotrexate, cyclosporine, a monoclonal antibody, pimecrolimus, tacrolimus, azathioprine, fluoruracil, salicylic acid, benzoyl peroxide, antibiotics or alpha-hydroxy acids.

Additional therapeutic agents include, for example, Tretinoin, retinol (cosmetic), Isotretinoin, Etretinate, Acitretin, steroids, hydrocortisone, hydrocortisone acetate, cortisone acetate, tixocortol pivalate, prednisolone, methylprednisolone, prednisone, betamethasone, betamethasone sodium phosphate, dexamethasone, dexamethasone sodium phosphate, fluocortolone, Clobetasol, Pathenol, triclosan, neomycin, tetracycline; ascorbic acid, vitamin E, pentapeptide, haloxyl, argireline, niacinamide, Retinyl Palmitate, Ascorbyl glucoside, Medicago sativa (Alfalfa) extract, Acer saccharum, Lupinus albus seed extract, Phyllanthus emblica fruit extract, dimethylmethoxy chromanol, Sophora angustifolia root extract, Ginkgo biloba leaf extract, N-hydroxysuccinimide, chlorhexidine digluconate, ascorbic acid, palmitoyl oligopeptide, palmitoyl tetrapeptide-7, Biotin and combinations thereof.

### The Color Corrective Composition

In at least one embodiment, the present teachings include a color corrective composition. The language “color corrective composition” includes a component that, when applied to the skin, changes the color of the skin. In one embodiment, the color corrective composition includes at least one foundation component and at least one coloring component and at least one stabilizer component.

One embodiment of the present teachings provide a color corrective composition comprising:
a) a foundation component; and
b) a coloring component; and
c) a stabilizer component, wherein the foundation and stabilizer components stabilize the coloring component in the composition and enhance the longevity of the color.

In a specific aspect of this embodiment, the color corrective composition is used in conjunction with a film-forming agent.

The term “film forming agent” refers to a composition or formulation that, when applied to the color corrective composition, forms an elastomeric film. The color corrective formula is either coated or incorporated into the film, or a combination of both. For example, the film forming agent may include a reactive reinforcing component and a cross-linking agent as described below. Representative film-forming agents are described below and in Example 2. Additional film-forming agents that can be used in this capacity are described in PCT/US2011/050003, published as WO2012/030984, the teachings of which are incorporated herein by reference.

The term “changing the color of skin” or “change the color of skin”, or “modulating the color of skin” refers to any change in skin color or skin tone. This change may be qualitative, as perceived by observers or it may also be quantitative when detected and/or measured by instruments, e.g., instruments capable of describing color spectrum. This term also refers to changes in appearance of skin imperfections that were present on the skin before application of color corrective composition, such as uneven skin tone, blotches, blemishes or discoloration. Representative means to change the color of skin can be found in the following examples, especially Examples 1 and 3-6.

The term “enhance the longevity of the color” means that, compared to traditional color corrective compositions, the color corrective compositions are longer lasting and require fewer repeated applications. The enhancement in longevity of color may be quantitative and may be measured by using laboratory tests, such as water immersion test. Representative means to establish the longevity of the color corrector can be found in the following examples, especially Examples 1 and 3-6.

The term “stabilize coloring component” or “stabilizing coloring component” refers to enhanced uniformity of color achieved by applying the color corrective composition described herein to the skin as compared to other color corrective compositions. The enhanced uniformity of color may be achieved immediately after application of the color corrective composition described herein to the skin and may last for a period of time, e.g., for 1, 2, 3, 4, 5, 6, 7, 8, 9, 10, 11, 12, 13, 14, 15, 16, 17, 18, 19, 20, 21, 22, 23 or 24 hours. The enhanced uniformity of color may result from the color component being prevented from precipitating or crashing out of the composition when the color corrective composition is applied to the skin. Stabilization of the uniformity of color are described in the following examples, especially Examples 1 and 3-6.

Another embodiment of the present teachings provides a color corrective composition comprising:
a) a foundation component; and
b) a coloring component; and
c) a stabilizer component,
d) a sun protection component, wherein the foundation and stabilizer components enhance the sun protective properties of the sun protection component.

In a specific aspect of this embodiment, the color corrective composition is used in conjunction with a film-forming agent. The term “enhance the sun protective properties” means that compared to traditional sunscreen, the substantivity of the claim composition is anticipated to increase. The increase thereby provides improved durability and coverage of the skin surface and are longer lasting and require fewer repeated applications. (See Examples 3 and 4, below). The enhancement in sun protective properties may be quantitative and may be measured by using laboratory tests.

The foundation component consists of a mixture of vinyl functionalized polysiloxanes and fumed silica. The foundation component provides stability to the overall composition and also provides a basis for producing a film if the optional step of adding a reactive reinforcing component and a cross-linking component is applied with or over the color corrective component. The combination is selected based on rheology and optionally, the film forming properties, necessary for effective SPF and pigment deposition on skin.

In one embodiment, the foundation component comprises vinyl dimethicone and silica silylate. In some embodiments, the vinyl dimethicone is comprised of a low viscosity vinyl dimethicone. In other embodiments, the vinyl dimethicone is comprised of a high viscosity vinyl dimethicone. In some embodiments, the vinyl dimethicone is a combination of a low viscosity vinyl dimethicone and a high viscosity vinyl dimethicone.

In some embodiments, the viscosity of the vinyl dimethicone is between about 1,000 Cp and about 40,000 Cp at 25° C. In some embodiments, the viscosity of the vinyl dimethicone is between 2,500 Cp and 30,000 Cp at 25° C. In some embodiments, the viscosity of the vinyl dimethicone is between 5,000 Cp and 20,000 Cp at 25° C. In other embodiments, viscosity of the vinyl dimethicone is between 100,000 Cp and 400,000 Cp at 25° C. In some embodiments, viscosity of the vinyl dimethicone is between 125,000 Cp and 300,000 Cp at 25° C. In other embodiments, viscosity of the vinyl dimethicone is between 150,000 Cp and 200,000 Cp at 25° C.

In some embodiments, the foundation component comprises vinyl dimethicone and silica silylate in a ratio of about 2:1. For example, in some embodiments, the foundation component comprises vinyl dimethicone and silica silylate in a ratio of about 1.95:1, about 1.9:1, about 1.85:1, about 1.8:1, about 1.75:1, about 1.7:1, about 1.65:1 or about 1.6:1.

In some embodiments, the foundation component comprises 1 to 30% of the composition. In certain embodiments, the foundation component comprises 10% of the composition.

The coloring component includes one or more pigments. These include natural or non-natural coloring agents or dyes. In one embodiment, the pigments are fluorescent dyes. In one embodiment, the pigment is an inorganic oxide. The inorganic oxide can be, for example, iron oxide, titanium dioxide, zinc oxide, mica, fluorophlogopite or a combination thereof.

In some embodiment, the pigment is dispersed in a pigment solubilizer. The pigment solubilizer allows for an even distribution of the pigment in the films and formulations. The pigment solubilizer is selected based on its ability to wet the pigments, disperse them effectively, dry quickly and deposit pigments evenly upon drying. The pigment solubilizer is utilized in combination and in proper amounts to achieve shades that span the human skin tone. Adjustments in total and relative concentration of pigment solubilizer as part of the color corrective formulation can yield a wide range of shades, color intensity, blemish coverage and naturalness of pigment appearance.

In some embodiments, the pigment solubilizer is a silicone fluid; hydrocarbon fluid; hydrohalocarbon fluid; a cosmetic ester; a cosmetic ether; an amino fluid; or a combination thereof. Such solubilizers are known to those of skill in the art. Non-limiting examples of cosmetic esters may include isopropyl palmitate, isopropyl myristate, isooctyl palmitate and others as supplied by Croda or Lubrizol (for example, Hydroxylated Lanolin; OHlan™ Lanolin Derivative Isocetyl Stearate; Schercemol™ ICS Ester Isodecyl Neopentanoate; Schercemol™ 105 Ester; Isodecyl Oleate; Schercemol™ IDO Ester; Isopropyl Isostearate; Schercemol™ 318 Ester). Cosmetic ethers have another functional group found either on silicone chains, or primarily in the glycol family. These provide enhance solubility and/or stability in the presence of any water. Oxiteno is supplier of such cosmetic ethers. Non-limiting examples of cosmetic ethers may include polyoxyethylene ethers; Methyl Glycol Monoethylene glycol methyl ether 2-methoxyethanol; Methyl Diglycol Diethylene glycol methyl ether; Ethyl Glycol Monoethylene glycol ethyl ether 2-ethoxyethanol; Ethyl Diglycol Diethylene glycol ethyl ether; Ethyl Triglycol Triethylene glycol ethyl ether; and Butyl Glycol Monoethylene glycol butyl ether 2-butoxyethanol. Amino fluids are siloxanes functionalized with amine groups, for example by Dow Corning. Non-limited examples may include Amino Silicone Emulsions; 949 Cationic Emulsion amino fluid cationic emulsion; 2-8194 Cationic Emulsion amino fluid cationic microemulsion; CE-8170 AF Microemulsion amino fluid non-ionic microemulsion; 5-7113 Silicone Quat Microemulsion Silicone quat non-ionic microemulsion; and CE 8401 Emulsion Amino polyether fluid non-ionic emulsion; or a combination thereof.

In some embodiments, the pigment solubilizer is selected from dimethicone; trisiloxane; methyl trimethicone; diphenylsiloxy phenyl trimethicone; caprylyl methicone; ethyl trisiloxane; isododecane; isohexadecane; isoeicosane; polyisobutene; disiloxane; methylpolysiloxane; octamethyltrisiloxane; decamethyltetrasiloxane; bis-hydroxyethoxypropyl dimethicone; bis-stearyl dimethicone; castor oil bis-hydroxypropyl dimethicone esters; trifluoromethyl C1-4 alkyl dimethicone; cyclopentasiloxane and combinations thereof. In other embodiments, the pigment solubilizer is selected from trisiloxane, dimethicone and combinations thereof.

In some embodiments, the selected inorganic oxides are surface modified to improve dispersion in low molecular viscosity (>100 cp) silicone fluid and reduce agglomeration in the dispersing fluid. For example, the oxides may be dispersed in Trisiloxane and Dimethicone at concentrations that optimize mixture rheology for oxide milling and for dispersion storage. In a specific embodiment, the pigment is a mixture of titanium oxide and Red, Yellow and Black iron oxides and is dispersed in the pigment solubilizer PMX 1184 (Trisiloxane (and) Dimethicone).

In some embodiments, the pigment and pigment solubilizer comprise 0.5% to 8% of the composition. In certain embodiments, the pigment and pigment solubilizer comprises 3% to 4% of the composition.

In some embodiments, the coloring component further comprises a pigment stabilizer. In some embodiments, the pigment stabilizer is selected from an alkyl dimethicone; an alkyl ester dimethicone; an alkyl ether dimethicone and combinations thereof. In certain embodiments, the pigment stabilizer is selected from PEG-12-dimethicone; PEG-11 methyl ether dimethicone; PEG/PPG-20/22 butyl ether dimethicone; PEG-9 dimethicone; PEG-10 dimethicone; PEG-9 polydimethylsiloxyethyl dimethicone; lauryl PEG-9 polydimethylsiloxyethyl dimethicone; dimethicone/PEG-10/15 crosspolymer; PEG-15/lauryl dimethicone crosspolymer; isododecane; PEG-17 dimethicone; bis-isobutyl PEG/PPG-10/7/dimethicone copolymer; PEG/PPG-19/19 dimethicone; C13-16 isoparafin; C10-13 isoparafin; PEG/PPG-18/18 dimethicone; lauryl PEG/PPG-18/18 methicone; caprylyl methicone; sorbitan monolaurate; sorbitan monolaurate/polysorbate 80; polysilicone-11, laureth-12 and combinations thereof. In one embodiment, the pigment stabilizer is PEG-12-dimethicone.

In some embodiments, the pigment stabilizer comprises 0.2% to 0.5% of the composition. For example, in one embodiment, the pigment stabilizer comprises 0.2% to 0.3% of the composition.

The stabilizer component provides significant optical, tactile and thickening properties to the formulation. The stabilizer component comprises elastomer beads, crosspolymers, or a combination thereof. In some embodiments, the stabilizer component comprises dimethicone crosspolymer; vinyldimethicone crosspolymer; dimethyl/methylhydrogen siloxane; bis-isobutyl PPG20 crosspolymer; PEG-10/15 crosspolymer; PEG-15/lauryl dimethicone crosspolymer; PEG-10/lauryl dimethicone crosspolymer; polyglycerin-3 crosspolymer; lauryl dimethicone; acrylates/dimethicone copolymer; cetearyl dimethicone/vinyl crosspolymer; C30-40 alkyl cetearyl dimethicone crosspolymer; ethyl trisiloxane; stearoxymethicone/dimethicone copolymer; polysilicone-11; Laureth-12; Dimethicone Crosspolymer; Dimethicone/Bis-Isobutyl PPG 20 Crosspolymer; Dimethicone/Vinyldimethicone Crosspolymer; or combinations thereof. In certain embodiments, the stabilizer component comprises dimethicone crosspolymer and vinyldimethicone crosspolymer.

In some embodiments, the stabilizer component comprises 30% to 70% of the composition. For example, in some embodiments, the stabilizer component comprises 45% to 55% of the composition.

In at least one embodiment, the color corrective composition further comprises a sun protection component; a sensory enhancing component; or a combination thereof.

In some embodiments, the color corrective composition further comprises a sun protection component. In certain embodiments, the sun protection component has a SPF functionality that is provided by including physical and or chemical UV absorbers. UV absorbers are selected in combination of FDA approved materials at concentration needed to achieve broad spectrum coverage as defined by the FDA monograph and estimated by BASF sunscreen simulator tool (See http://www.sunscreensimulator.basf.com/Sunscreen_Simulator/, the teachings of which hare incorporated herein by reference). In one embodiment, the sun protection component comprises a sunscreen. The sunscreen can comprise, for example, an organic sunscreen; an inorganic sunscreen; or a combination thereof.

The selection of the sunscreen used in the sun protection component should be balanced to achieve the desired SPF UV protection while not compromising the other performance attributes of color corrective composition. The cocktail contributes to formula durability on skin, formula rheology and optical properties of formulation on skin. A representative sun protection component is 79-205, which provides SPF 15 while achieving appropriate opacity on skin, and also provides sufficient blemish coverage with natural color deposition.

In particular embodiments, the inorganic sunscreen is an inorganic oxide. For example, the inorganic oxide can be titanium dioxide, zinc oxide, iron oxides or combinations thereof. In one embodiment, the inorganic oxide is zinc oxide. Representative iron oxides, include e.g., ferric oxide hydrate [FeO(OH)], ferric oxide (FeO₃) or ferric ferrous oxide (Fe₃O₄).

Optionally, the inorganic oxide is dispersed in cyclopentasiloxane. Alternatively, the inorganic oxide is dispersed in a Silicone Fluid; Hydrocarbon Fluid; Hydrohalocarbon Fluid; cosmetic ester; a cosmetic ether; or an amino fluid as described above. Specific non-limiting examples include Dimethicone; Methyl Trimethicone; Diphenylsiloxy Phenyl Trimethicone; Caprylyl Methicone; Ethyl Trisiloxane; Isododecane; Isohexadecane; Isoeicosane; Polyisobutene; Disiloxane; Methylpolysiloxane; Octamethyltrisolxane; Decamethyltetrasiloxane; Bis-Hydroxyethoxypropyl Dimethicone; Bis-stearyl Dimethicone; Castor Oil Bis-Hydroxypropyl Dimethicone Esters; and Trifluoromethyl C1-4 alkyl dimethicone.

In particular embodiments, the organic sunscreen is octinoxate; octocrylene; ethylhexyl methoxycrylene (solastay S1); Ecamsule (Mexoryl SX, Terephthalylidene Dicamphor Sulfonic Acid); Avobenzone (1-(4-methoxyphenyl)-3-(4-tert-butyl phenyl)propane-1,3-dione, Butyl methoxy dibenzoylmethane, BMDBM, Parsol 1789, Eusolex 9020); Trolamine salicylate (Triethanolamine salicylate); Sulisobenzone (2-Hydroxy-4-Methoxybenzophenone-5-sulfonic acid,3-benzoyl-4-hydroxy-6-methoxybenzenesulfonic acid, Benzophenone-4, Escalol 577); Octyl salicylate (Octisalate, 2-Ethylhexyl salicylate, Escalol 587); Octocrylene (Eusolex OCR, 2-cyano-3,3diphenyl acrylic acid, 2-ethylhexylester); Menthyl anthranilate (Meradimate); Homosalate (Homomethyl salicylate, HMS); Oxybenzone (Benzophenone-3, Eusolex 4360, Escalol 567); Dioxybenzone (Benzophenone-8); Cinoxate2 (Ethoxyethyl p-methoxycinnamate); Phenylbenzimidazole sulfonic acid (Ensulizole, Eusolex 232, PBSA, Parsol HS); Padimate-(O OD-PABA, octyldimethyl-PABA, σ-PABA); p-Aminobenzoic acid (PABA); or a combination thereof. In certain embodiments, the organic sunscreen is selected from octinoxate; avobenzone; octocrylene; solastay S1; and combinations thereof.

In at least one embodiment, the sunscreen comprises octinoxate; avobenzone; octocrylene; methoxycrylene; titanium dioxide; zinc oxide or a combination thereof, wherein the titanium dioxide and the zinc oxide are optionally dispersed in cyclopentasiloxane.

In at least one embodiment, the color corrective composition further comprises a sensory component. The sensory component further defines the optics and texture of the color corrective composition. In some embodiments, the sensory enhancing component comprises elastomer beads, inorganic powders, organic beads; or a combination thereof. In certain embodiments, the sensory enhancing component comprises polymethyl methacrylate; methylmethacrylate crosspolymer; acrylates crosspolymer; styrene/DVB copolymer; polymethylsilsesquioxane; vinyl dimethicone crosspolymer; methicone silsesquioxane crosspolymer; diphenyl dimethicone/vinyl diphenyl dimethicone/silsesquioxane crosspolymer; boron nitride; nylon 6; nylon 12; isopropyl titanium triisostearate; alumina; polymethylsilsesquixoane; hdi/trimethylol hexyllacetone crosspolymer; polyethylene; silica; ethylene/acrylic acid copolymer; polylactic acid; polyethylene; cellulose; titanium dioxide; aluminum hydroxide; or a combination thereof. In some embodiments, the sensory enhancing component is Nylon 12 and isopropyl titanium triisostearate.

In at least one embodiment, the sensory enhancing component comprises 0% to 10% of the composition. In a particular embodiment, the sensory enhancing comprises 4% to 5% of the composition.

In a specific embodiment, the sensory properties of the formulation are further adjusted by selection of volatile and non-volatile fluids which control dry time and pigment depositions. The formulations, for example, can contain primarily highly volatile cyclomethicones, trisiloxane and short (<6cP) dimethicone silicones with some high molecular weight dimethicones and in some cases water. The solvents are included neat or as part of other components. In a particular embodiment, dimethicone and trisiloxane (PMX 1184) is added with dispersed pigments, cyclopentasiloxane is added with zinc oxide, and DC 9045 elastomer. The DC 9045 elastomer provides significant optical, tactile and thickening properties to the formulation. The optics and texture of the color corrective composition is further defined by Nylon 12 (And) Isopropyl Titanium Triisostearate (Nylon 10-I2™). Nylon modified with Isopropyl Titanium Triisostearate provides unique texture, optics and miscibility with the other formulation components.

### Films

In one embodiment, a benefit-inducing film is prepared by a process comprising the steps of:

a) applying a benefit-inducing layer to the skin;

b) applying a reactive reinforcing component to said benefit-inducing layer; and

c) applying a reactive reinforcing component to said reactive reinforcing component,

wherein said cross-linking component facilitates in situ cross-linking of the reactive reinforcing component and the benefit-inducing layer, thereby enhancing or providing a benefit to skin.

In one embodiment, a color corrective film prepared by a process comprising the steps of:
a) applying a color corrective composition;
b) applying a reactive reinforcing component to skin; and
c) applying a cross-linking component to said reactive reinforcing component, wherein said cross-linking component facilitates in situ cross-linking of the reactive reinforcing component and the color corrective composition, such that a color corrective film is formed on skin.

In specific aspects of this embodiment, the color corrective formulation is as described in the color corrective section above.

### Methods of Use

One embodiment of the present teachings provides a method of enhancing or providing a benefit to skin comprising the steps of:

a) applying a benefit-inducing layer to the skin;

b) applying a reactive reinforcing component to said benefit-inducing layer; and

c) applying a reactive reinforcing component to said reactive reinforcing component,

wherein said cross-linking component facilitates in situ cross-linking of the reactive reinforcing component and the benefit-inducing layer, thereby enhancing or providing a benefit to skin.

The language “benefit-inducing layer” refers to a layer comprising an active ingredient that can impart a benefit to the skin. In some embodiments, the benefit inducing layer may be a color corrective composition. In further embodiments, the color corrective composition may also comprise one or more therapeutic agents. In some embodiments, the therapeutic agent may be a moisturizer, mineral oil, petroleum jelly, coal tar, anthralin, a corticosteroid, fluocinonide, vitamin D₃ analogues, retinoids, phototherapy, methotrexate, cyclosporine, a monoclonal antibody, pimecrolimus, tacrolimus, azathioprine, fluoruracil, salicylic acid, benzoyl peroxide, antibiotics or alpha-hydroxy acids.

Additional active ingredients include antiaging/acne retinoids; eczema or psoriasis treatments; treatments for wound healing; antibacterials; and/or cosmetic ingredients. For example, a non-limiting list of active ingredients includes Tretinoin, retinol (cosmetic), Isotretinoin, Etretinate, Acitretin, steroids, hydrocortisone, hydrocortisone acetate, cortisone acetate, tixocortol pivalate, prednisolone, methylprednisolone, prednisone, Betamethasone, betamethasone sodium phosphate, dexamethasone, dexamethasone sodium phosphate, fluocortolone, Clobetasol, Pathenol, triclosan, neomycin, tetracycline, ascorbic acid, vitamin E, pentapeptide, haloxyl, argireline, niacinamide, Retinyl Palmitate, Ascorbyl glucoside, Medicago sativa (Alfalfa) extract, Acer saccharum, Lupinus albus seed extract, Phyllanthus emblica fruit extract, dimethylmethoxy chromanol, Sophora angustifolia root extract, Ginkgo biloba leaf extract, N-hydroxysuccinimide, chlorhexidine digluconate, ascorbic acid, palmitoyl oligopeptide, palmitoyl tetrapeptide-7, Biotin, and combinations thereof.

In yet another embodiment, the benefit-inducing layer comprises one or more therapeutic agents as defined above but does not comprise color corrective composition.

One embodiment provides a method for changing the color of skin, comprising applying to skin a composition comprising the color corrective composition described above.

Certain embodiments provide a method of changing the color of skin comprising the steps of:
a) applying a color corrective composition to the skin;
b) applying a reactive reinforcing component to color corrective composition; and
c) applying a cross-linking component to said reactive reinforcing component, wherein said cross-linking component facilitates in situ cross-linking of the reactive reinforcing component and the color corrective composition, thereby changing the color of the skin.

In specific aspects of this embodiment, the color corrective formulation is as described in the color corrective section above.

The language “skin or body imperfections” include those items on a subject's skin that the subject perceives as a blemish or a flaw. Examples of skin imperfections include, but are not limited to, port wine stain or nevus flammeus (e.g., nevus flammeus nuchae or midline nevus flammeus) melasma, wrinkles, blemishes, acne, moles, scars, tattoos, bruises, skin disfigurements, birth marks, sun damage, age damage, uneven skin tone, sagging skin, skin roughness, hyperpigmentation, enlarged pores, telangiectasia, redness, shine, cellulite, stretch marks or loss of skin elasticity.

The compositions, films and methods disclosed herein are useful in changing the color of skin that is wounded, affected by a dermatological disorder, or has been treated by a laser.

“Wounded skin” or “wounds” includes injuries to the skin wherein the skin is torn, cut or punctured. A wound is a break in the skin. In one embodiment, the wound is caused by skin contact with a foreign object. The break in the skin may cause external bleeding. Wounds include open wounds, for example, abrasions, lacerations, incisions, punctures, avulsions, or amputations. Wounds also include burn wounds. A burn is a type of injury to flesh caused by heat, electricity, chemicals, light, radiation or friction.

The language “dermatological disorder” includes disorders that cause at least one symptom on the skin of a subject requiring medical treatment. In one embodiment, dermatological disorders are caused by autoimmune disorders. In another embodiment, a dermatological disorder is caused by environmental factors, such as allergens or chemicals. Examples of symptoms of dermatological disorders requiring treatment include dermatitis, itchy skin, dry skin, crusting, blistering, or cracking skin, skin edema, or skin lesion formation. Dermatological disorders include, but are not limited to, lichen simplex chronicus, cutaneous lupus (e.g., acute cutaneous lupus, subacute cutaneous lupus, chronic cutaneous lupus, chilblain lupus erythematosus, discoid lupus erythematosus, lupus erythematosus-lichen planus overlap syndrome, lupus erythematosus panniculitis, tumid lupus erythematosus and verrucous lupus erythematosus), psoriasis (e.g., psoriasis vulgaris, psoriatic erythroderma, pustular psoriasis, drug-induced psoriasis, inverse psoriasis, seborrheic-like psoriasis and guttate psoriasis), eczema (e.g., atopic eczema, atopic dermatitis, contact dermatitis, xerotic eczema, seborrhoeic dermatitis, dyshidrosis, discoid eczema, venous eczema, dermatitis herpetiformis, neurodermatitis and autoeczematization), and chronic dry skin. In at least one embodiment, the dermatological disorder is lichen simplex chronicus, cutaneous lupus, psoriasis, eczema, or chronic dry skin. In a specific embodiment, the dermatological disorder is psoriasis. In addition, dermatological disorders also include ichthyosis, rosacea and xeroderma. In a specific embodiment, the dermatological disorder is xeroderma, eczema, psoriasis, rosacea or ichthyosis. In a particular embodiment, the dermatological disorder is xeroderma, atopic dermatitis, psoriasis or ichthyosis. In a particular embodiment, the dermatological disorder is an ulcer.

The term “treated by a laser” includes any procedure performed by exposing the subject's skin or body to a laser. The laser treatment may be ablative or non-ablative. Representative laser treatments include laser therapies for cosmetic uses or for medical uses, and include, for example, skin rejuvenation; skin resurfacing; stretch marks; scar removal; wrinkle removal or reduction; leg vein or artery removal; tattoo removal; removal of stretch marks; removal of sunspots; removal of birthmarks; telangiectasia; rosacea; angiomas; hemangiomas; reticular veins; port wine stains; liposuction; hair removal, removal of precancerous lesions, and skin cancer surgery.

The term “exposed to a laser” means a laser light was shone onto the subject's skin or body.

The term “post-laser treatment” means that the subject has undergone a laser treatment prior to treatment with the compositions, formulations, films and methods described herein.

The term “body” includes any part of the subject's body that can benefit from the formulations disclosed herein. Examples of the subject's body include the skin, the neck, the brow, the jowls, the eyes, the hands, the feet, the face, the cheeks, the breasts, the abdomen, the buttocks, the thighs, the back, the legs, the ankles, cellulite, fat deposits, and the like.

The term “skin” includes the epidermis of the subject's skin, which is the outer layer of the skin and includes the stratified squamous epithelium composed of proliferating basal and differentiated suprabasal keratinocytes.

The term “subject” includes subjects in which the compositions disclosed herein would be appropriate for use. In one embodiment, the subject is a mammal, for example, a human.

The terms “apply,” “applied” and “application” includes methods of contacting or administering the composition or formulation disclosed herein to a subject's skin or body, such as application by fingers, brush, cotton ball, pad, spray, sponge, cotton swab, roll-on and the like. One of skill in the art can readily determine appropriate methods to apply the compositions disclosed herein.

In using the compositions disclosed herein, one would first identify the area on the body or skin that may benefit from use of the compositions disclosed herein. A pre-treatment of the area (for example, washing, shaving, or otherwise preparing the area) may be completed, if necessary. The amount of the composition applied is determined by the size and location of the area to be treated as well as the skin condition and type. After the optional pretreatment, the composition is applied. After the composition is applied, a further step of applying the reactive reinforcing component and the crosslinking component to the area either sequentially or in combination to form the film over the entire or over a portion of the area. The amount of both the reactive reinforcing component and/or the crosslinking component is determined by the size and location of the area to be treated as well as the skin condition and type. The film may be left over the area for a period of time as determined by the subject. The film can be removed by use of the film removing cleanser as described herein as PCT/US2011/050003, published as WO2012/030984, the teachings of which are incorporated herein by reference. The treatment can be repeated as many times as needed in order to achieve a desired result.

The language “formulation” and “composition” are used interchangeably and include a composition (or formulation) that, when applied to the body or skin of a subject, forms a film on the body resulting in a benefit to the subject. Benefits include, but are not limited to, long lastingness of color correction, long lasting broad-spectrum SPF delivery, adjustable pigment smoothness, and adjustable tone to hide skin imperfections, wounds or dermatological disorders (for example, dark circles, age spots, ache, blood vessels, rosacea, etc.).

In some embodiments, the present teachings pertain, at least in part, to a kit comprising a color corrective composition. In some embodiments, the kit further comprises instructions for use of the kit, one or more brushes, one or more swabs, a film removing cleanser or a mirror. In some embodiments, the kit further comprises one or more finishing formulations.

In some embodiments, the present teachings pertain, at least in part, to a film removing cleanser for use in removing the color corrective film, wherein said film is prepared by a process comprising the steps of a) applying a reactive reinforcing component to skin; and b) applying a cross-linking component to said reactive reinforcing component, wherein said cross-linking component catalyzes an in situ cross-linking of the reactive reinforcing component. The film removing cleanser is described in PCT/US2011/050003, published as WO2012/030984, the teachings of which are incorporated herein by reference.

In at least one embodiment, the present teachings include a reactive reinforcing component and a cross-linking component. The language “reactive reinforcing component” includes a component that, when applied to the skin after the application of the color corrective composition, is the basis of the film that is formed upon application of the cross-linking component to the reactive reinforcing component. In one embodiment, the reactive reinforcing component includes at least one reactive constituent and at least one reinforcing constituent.

The language “reactive constituent” includes one or more constituents of the reactive reinforcing component that provide the reactive film-forming elements of the formulation. In some embodiments, the reactive constituent includes at least one polysiloxane, polyethylene oxide, polypropylene oxide, polyurea, polyurethane, polyester (including polylactic-co-glycolic acid, polycaprolactone, polylactic acid, polyglycolic acid, and polyhydroxybutyrate, polyamide, or polysulfone. In another embodiment, the reactive constituent is a compound of formula I:

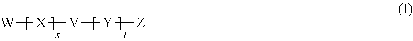

wherein:

W is R¹R²R³SiO—, —OR⁴, —NR⁵R⁶, —CR⁷R⁸R⁹ or C₅₋₁₀ aryl;

X is —R¹¹R¹²Si—O—, —OCONR¹³—, —NR¹⁴CONR¹⁵—, —CO—, —NR¹⁶CO—, —SO₂—, —O—, —S— or —NR¹⁷—;

V is absent, C₁₋₂₀ alkyl, C₂₋₂₀ alkenyl, C₅₋₁₀ aryl, —O—, —NR¹⁰— or —S—;

Y is —R¹⁸R¹⁹Si—O—, —OCONR²⁰—, —NR²¹CONR²²—, —CO—, —NR²³CO—, —SO₂—, —O—, —S— or —NR²⁴;

Z is —SiR²⁵R²⁶R²⁷, —OR²⁸, —NR²⁹R³⁰, —CR³¹R³²R³³ or C₅₋₁₀ aryl; R¹, R², R³, R⁷, R⁸, R⁹, R¹¹, R¹², R¹⁸ R¹⁹, R²⁵, R²⁶, R²⁷, R³¹, R³² and R³³ are each independently hydrogen, C₁₋₂₀ alkyl, C₂₋₂₀ alkenyl, C₅₋₁₀ aryl, hydroxyl or C₁₋₂₀ alkoxyl;

R⁴, R⁵, R⁶, R¹³, R¹⁴, R¹⁵, R¹⁶, R¹⁷, R²⁰, R²¹, R²², R²³, R²⁴, R²⁸, R²⁹ and R³⁰ are each independently hydrogen, C₁₋₂₀ alkyl, C₂₋₂₀ alkenyl, C₅₋₁₀ aryl; and

s and t are each independently an integer from about 0 to about 6000.

X and Y of formula I represent an independent “monomer unit.” The number of X and Y monomer units present in formula I is provided by the value of s and t, respectively. Representative monomer units include:

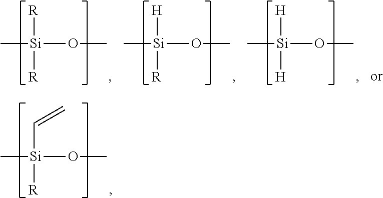

where R is as for defined for R¹, R², R³, etc, above.

It is understood that when more than one X (or Y) monomer unit is present (e.g. s (or t) is more than one), the values for R¹¹, R¹², R¹³, R¹⁴, R¹⁵, R¹⁶, R¹⁷, R¹⁸, R¹⁹, R²⁰, R²¹, R²², R²³, and R²⁴ are selected independently for each individual monomer unit described by —[X]ₛ— (or —[Y]ₜ—). For example, if the value of the monomer unit X is —R¹¹R¹²Si—O— and the value of s is 3, then —[X]ₛ— is:

### —[R¹¹R¹²Si—O—R¹¹R¹²Si—O]—.

In this example, it is understood that the three R¹¹ groups present in may be the same or different from each other, for example, one R¹¹ may be hydrogen, and the two other R¹¹ groups may be methyl.

W and Z of formula I represent independent terminal caps, one on each end of the polymer. For example, terminal caps include:

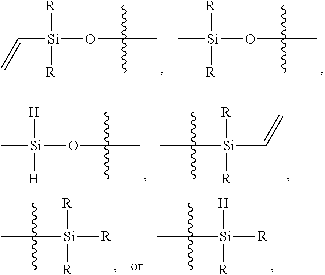

wherein [symbol] denotes attachment to a monomer unit and wherein R is as for defined for R¹, R², R³, etc, above.

In one embodiment,

W is R¹R²R³SiO—, —OR⁴, —NR⁵R⁶, —CR⁷R⁸R⁹ or C₅₋₁₀ aryl;

X is —R¹¹R¹²Si—O—, or —NR¹⁴CONR¹⁵—;

V is absent, C₁₋₂₀ alkyl, C₂₋₂₀ alkenyl, C₅₋₁₀ aryl, —O—, —NR¹⁰— or —S—;

Y is —R¹⁸R¹⁹Si—O—, or —NR²¹CONR²²—;

Z is —SiR²⁵R²⁶R²⁷, OR²⁸, —NR²⁹R³⁰, —CR³¹R³²R³³ or C₅₋₁₀ aryl;

R¹, R², R³, R⁷, R⁸, R⁹, R¹¹, R¹², R¹⁸, R¹⁹, R²⁵, R²⁶, R²⁷, R³¹, R³² and R³³ are each independently hydrogen, C₁₋₂₀ alkyl, C₂₋₂₀ alkenyl, C₅₋₁₀ aryl, hydroxyl or C₁₋₂₀ alkoxyl;

R⁴, R⁵, R⁶, R¹⁴, R¹⁵, R²¹, R²², R²⁸, R²⁹ and R³⁰ are each independently hydrogen, C₁₋₂₀ alkyl, C₂₋₂₀ alkenyl, C₅₋₁₀ aryl; and

s and t are each independently an integer from about 0 to about 6000, wherein the sum of and t is not 0.

In one embodiment,

W is R¹R²R³SiO—, —CR⁷R⁸R⁹ or C₅₋₁₀ aryl;

X is —R¹¹R¹²Si—O—, or —NR¹⁴CONR¹⁵—;

V is absent, C₁₋₂₀ alkyl, C₂₋₂₀ alkenyl, or C₅₋₁₀ aryl;

Y is —R¹⁸R¹⁹Si—O—, or —NR²¹CONR²²—;

Z is —SiR²⁵R²⁶R²⁷, —CR³¹R³²R³³ or C₅₋₁₀ aryl;

R¹, R², R³, R⁷, R⁸, R⁹, R¹¹, R¹², R¹⁸, R¹⁹, R²⁵, R²⁶, R²⁷, R³¹, R³² and R³³ are each independently hydrogen, C₁₋₂₀ alkyl, C₂₋₂₀ alkenyl, C₅₋₁₀ aryl, hydroxyl or C₁₋₂₀ alkoxyl;

R¹⁴, R¹⁵, R²¹, and R²² are each independently hydrogen, C₁₋₂₀ alkyl, C₂₋₂₀ alkenyl, C₅₋₁₀ aryl; and

s and t are each independently an integer from about 0 to about 6000, wherein the sum of s and t is not 0.

In one embodiment, V is absent, W is R¹R²R³SiO—; X is —R¹¹R¹²Si—O—; Y is —R¹⁸R¹⁹Si—O—; Z is —SiR²⁵R²⁶R²⁷; and R¹, R², R³, R¹¹, R¹², R¹⁸, R¹⁹, R²⁵, R²⁶ and R²⁷ are each independently selected from C₁₋₂₀ alkyl (e.g., C₁ alkyl, such as methyl) or C₂₋₂₀ alkenyl (e.g., C₂ alkenyl, such as vinyl). In one embodiment, at least one of R¹, R², R³, R¹¹, R¹², R¹⁸, R¹⁹, R²⁵, R²⁶ and R²⁷ are C₂₋₂₀ alkenyl, for example, C₂ alkenyl (e.g., vinyl). In another embodiment, at least two of R¹, R², R³, R¹¹, R¹², R¹⁸, R¹⁹, R²⁵, R²⁶ and R²⁷ are C₂₋₂₀ alkenyl, for example, C₂ alkenyl (e.g., vinyl). In some embodiments, at least one of R¹, R², R³, R²⁵, R²⁶ and R²⁷ are each C₂₋₂₀ alkenyl, for example, C₂ alkenyl (e.g., vinyl).

In one embodiment, V is absent, W is R¹R²R³SiO—; X is —R¹¹R¹²Si—O—; Y is —R¹⁸R¹⁹Si—O—; Z is —SiR²⁵R²⁶R²⁷; and R¹, R², R³, R²⁵, R²⁶ and R²⁷ are each independently selected from C₁₋₂₀ alkyl (e.g., C₁ alkyl, such as methyl) or C₂₋₂₀ alkenyl (e.g., C₂ alkenyl, such as vinyl); and R¹¹, R¹², R¹⁸, and R¹⁹ are each independently selected from C₁₋₂₀ alkyl (e.g., C₁ alkyl, such as methyl). In one embodiment, at least one of R¹, R², R³, and at least one of R²⁵, R²⁶ and R²⁷ is C₂₋₂₀ alkenyl, for example, C₂ alkenyl (e.g., vinyl). In one embodiment, one of R¹, R², R³ is C₂ alkenyl (e.g., vinyl) and the others are C₁₋₂₀ alkyl (e.g., C₁ alkyl, such as methyl), and at least one of R²⁵, R²⁶ and R²⁷ is C₂₋₂₀ alkenyl, for example, C₂ alkenyl (e.g., vinyl) and the others are C₁₋₂₀ alkyl (e.g., C₁ alkyl, such as methyl). In one embodiment, at least one of R¹¹ or R¹² and at least one of R¹⁸ or R¹⁹ is C₂₋₂₀ alkenyl, for example, C₂ alkenyl (e.g., vinyl) for at least one monomer unit. In one embodiment, one of R¹¹ or R¹² is C₂ alkenyl (e.g., vinyl) and the others are C₁₋₂₀ alkyl (e.g., C₁ alkyl, such as methyl), and at least one of R¹⁸ or R¹⁹ is C₂₋₂₀ alkenyl, for example, C₂ alkenyl (e.g., vinyl) and the others are C₁₋₂₀ alkyl (e.g., C₁ alkyl, such as methyl) for at least one monomer unit.

In some embodiments, the organopolysiloxane includes vinyl moieties only at the terminal caps of the polymer. In some embodiments, the organopolysiloxane include vinyl moieties only in the monomer units, but not at the terminal cap of the polymer. In other embodiments, the organopolysiloxane includes vinyl moieties at both the terminal cap or in the monomer unit of the polymer. In one embodiment, the polymer includes two vinyl moieties located either at the terminal cap, or within the monomer unit, or a combination thereof. In at least one embodiment, the organopolysiloxane includes vinyl moieties only at the terminal caps of the polymer and contains Si—H units only within the monomer units and not at the terminal caps.

In one embodiment, on average at least two vinyl moieties are present in the polymer. In a specific embodiment, at least two vinyl moieties are present in the polymer and at least two vinyl moieties are present on the two terminal caps of the polymer. In a specific embodiment, only two vinyl moieties are present in the polymer. In a specific embodiment, only two vinyl moieties are present in the polymer and are located on each of the terminal caps. In a specific embodiment, on average at least two vinyl moieties are present in the polymer and at least two vinyl moieties are present in one or more monomer units of the polymer. In a specific embodiment, at least two vinyl moieties are present anywhere in the polymer, but separated from another vinyl moiety by about 2000 monomer units, for example, 1500, 1600, 1700, 1800, 1900, 2000, 2100, 2200, 2300, 2400, or 2500 monomer units. In a specific embodiment, on average at least two vinyl moieties are present anywhere in the polymer, but separated from another vinyl moiety by about 850 monomer units, for example, 350, 450, 550, 650, 750, 850, 950, 1050, 1150, 1250, or 1350 monomer units. In a specific embodiment, on average greater two vinyl moieties are present anywhere in the polymer, but separated from another vinyl moiety by about 40 monomer units, for example, 5, 10, 15, 20, 25, 30, 35, 40, 45, 50, 55, 60, 65, 70, 75, or 80 monomer units. In a specific embodiment, one or more Si—H units are present in addition to the vinyl moiety. Alternatively, in one embodiment, if a vinyl moiety is present then a Si—H is not present.

In one embodiment, V is absent, W is R¹R²R³SiO—; X is —R¹¹R¹²Si—O—; Y is —R¹⁸R¹⁹Si—O—; Z is —SiR²⁵R²⁶R²⁷; R¹, R², R³, R¹¹, R¹², R¹⁸, R¹⁹, R²⁵, R²⁶ and R²⁷ are each independently selected from hydrogen or C₁₋₂₀ alkyl (e.g., C₁ alkyl, such as methyl). In one embodiment, R¹, R², R³, R²⁵, R²⁶ and R²⁷ are each independently selected from C₁₋₂₀ alkyl (e.g., C₁ alkyl, such as methyl); and R¹¹, R¹², R¹⁸, and R¹⁹ are each independently selected from hydrogen or C₁₋₂₀ alkyl (e.g., C₁ alkyl, such as methyl), wherein at least one of and R¹¹, R¹², R¹⁸, and R¹⁹ are hydrogen for at least one monomer unit. In one embodiment, on average greater than two Si—H units (e.g. one or more of R¹¹, R¹², R¹⁸, and R¹⁹ is hydrogen) are present in the polymer, for example 3-15 Si—H units may be present. In a specific embodiment, 8 Si—H units are present on average. In one embodiment, one or more Si—H units (e.g. one or more of R¹¹, R¹², R¹⁸, and R¹⁹ is hydrogen) are present in the polymer. In one embodiment, at least two monomer units on average include a —Si—H unit (e.g. one or more of R¹¹, R¹², R¹⁸, and R¹⁹ is hydrogen). In one embodiment, at least three monomer units on average include a —Si—H unit (e.g. one or more of R¹¹, R¹², R¹⁸, and R¹⁹ is hydrogen). In one embodiment, at least four monomer units on average include a —Si—H unit (e.g. one or more of R¹¹, R¹², R¹⁸, and R¹⁹ is hydrogen). In one embodiment, at least five monomer units on average include a —Si—H unit (e.g. one or more of R¹¹, R¹², R¹⁸, and R¹⁹ is hydrogen). In one embodiment, at least six monomer units on average include a —Si—H unit (e.g. one or more of R¹¹, R¹², R¹⁸, and R¹⁹ is hydrogen). In one embodiment, at least seven monomer units on average include a —Si—H unit (e.g. one or more of R¹¹, R¹², R¹⁸, and R¹⁹ is hydrogen). In one embodiment, at least eight monomer units on average include a —Si—H unit (e.g. one or more of R¹¹, R¹², R¹⁸, and R¹⁹ is hydrogen). In one embodiment, a Si—H unit may be present in one or both the terminal caps in addition to being present in a monomer unit as described above. In one embodiment, one or more Si—H units may be present only in a monomer unit as described above, and not present in either of the terminal caps. In a specific embodiment, Si-(alkyl) or Si-(vinyl) units may also be present in the polymer. In a specific embodiment, only Si—CH₃ and Si—H units are present. In a specific embodiment, monomer units or terminal caps include C₁-C₂₀alkyl, e.g., methyl groups, for the non-Si—H positions of the polymer.

In a specific embodiment, on average at least two Si—H units are present in the polymer. In a specific embodiment, on average at least two Si—H moieties are present anywhere in the polymer, but separated from another Si—H moiety by about 2000 monomer units, for example, 1500, 1600, 1700, 1800, 1900, 2000, 2100, 2200, 2300, 2400, or 2500 monomer units. In a specific embodiment, on average at least two Si—H moieties are present only in the monomer units of the polymer and not the terminal cap, and are separated from another Si—H moiety by about 2000 monomer units, for example, 1500, 1600, 1700, 1800, 1900, 2000, 2100, 2200, 2300, 2400, or 2500 monomer units. In a specific embodiment, on average at least two Si—H units are present anywhere in the polymer, but separated from another Si—H moiety by about 850 monomer units, for example, 350, 450, 550, 650, 750, 800, 850, 950, 1050, 1150, 1250, or 1350 monomer units. In a specific embodiment, on average at least two Si—H moieties are present only in the monomer units of the polymer and not the terminal caps, and are separated from another Si—H moiety by about 2000 monomer units, for example, 350, 450, 550, 650, 750, 800, 850, 950, 1050, 1150, 1250, or 1350 monomer units. In a specific embodiment, on average greater than two Si—H units are present anywhere in the polymer, but separated from another Si—H moiety by about 40 monomer units, for example, 5, 10, 15, 20, 25, 30, 35, 40, 45, 50, 55, 60, 65, 70, 75, or 80 monomer units. In a specific embodiment, on average at least two Si—H moieties are present only in the monomer units of the polymer and not the terminal caps, and are separated from another Si—H moiety by about 2000 monomer units, for example, 5, 10, 15, 20, 25, 30, 35, 40, 45, 50, 55, 60, 65, 70, 75, or 80 monomer units.

In one aspect of any one of the above embodiments, the sum of s and t is an integer from about 1000 to about 8000; from about 1300 to about 2700; from about 1500 to about 2700; from about 1600 to about 2600; from about 1600 to about 2500; from about 1700 to about 2500; from about 1800 to about 2400; from about 1800 to about 2300; from about 1900 to about 2300; from about 2000 to about 2200; from about 2050 to about 2150; from about 2100.

In one aspect of any one of the above embodiments, the sum of s and t is an integer from about 200 to about 1100; from about 600 to about 1100; from about 700 to about 1000; from about 800 to about 900; from about 825 to about 875; from about 850; from about 200 to about 800; from about 225 to about 700; from about 250 to about 600; from about 275 to about 500; from about 300 to about 400; from about 350 to about 400; from about 375. In a specific embodiment, the sum of s and t is an integer from about 850.

In one aspect of any one of the above embodiments, the sum of s and t is an integer from about 5 to about 1300; from about 10 to about 1100; from about 10 to about 600; from about 15 to about 500; from about 15 to about 400; from about 20 to about 300; from about 20 to about 200; from about 25 to about 100; from about 25 to about 75; from about 30 to about 50; from about 40.

In some embodiments, the reactive constituent comprises at least one organopolysiloxane. The term “organopolysiloxane” includes compounds of formula II:

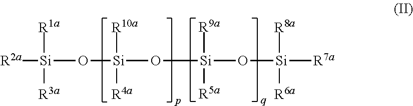

wherein R^{1a}, R^{3a}, R^{4a}, R^{5a}, R^{6a}, R^{7a}, R^{8a}, R^{9a} and R^{10a} are each independently selected from hydrogen, C₁₋₂₀ alkyl, C₂₋₂₀ alkenyl, C₅₋₁₀ aryl, hydroxyl or C₁₋₂₀ alkoxyl and p and q are each independently an integer from between 10 and about 6000.

In some embodiments, the organopolysiloxane is a compound of formula IIa:

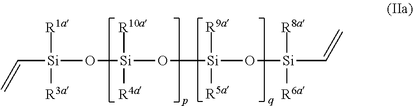

wherein R^{1a′}, R^{3a′}, R^{4a′}, R^{5a′}, R^{6a′}, R^{8a′}, R^{9a′} and R^{10a′} are each independently selected from hydrogen, C₁₋₂₀ alkyl, C₂₋₂₀ alkenyl, C₅₋₁₀ aryl, hydroxyl or C₁₋₂₀ alkoxyl and p and q are each independently an integer from between 10 and about 6000. In one embodiment, R^{1a′}, R^{3a′}, R^{4a′}, R^{5a′}, R^{6a′}, R^{8a′}, R^{9a′} and R^{10a′} are alkyl (e.g., C₁ alkyl, such as methyl).

The term “alkyl” includes both branched and straight-chain saturated aliphatic hydrocarbon groups having the specified number of carbon atoms. The term “C₁₋₂₀ alkyl” includes branched and straight chain aliphatic groups having between 1 and 20 carbons. Examples of alkyl moieties include methyl, ethyl, n-propyl, i-propyl, n-butyl, s-butyl, t-butyl, n-pentyl, n-hexyl, isopropyl, isobutyl, sec-butyl, tert-butyl, isopentyl, and s-pentyl. Moreover, the term alkyl includes both “unsubstituted alkyls” and “substituted alkyls,” the latter of which refers to alkyl moieties having substituents (e.g., F, Cl, Br, I, NO₂, CN, alkyl, aryl, hydroxyl, alkoxy, COCH₃ and the like) replacing a hydrogen on one or more carbons of the hydrocarbon backbone.

The term “alkenyl” includes the specified number of hydrocarbon atoms in either straight or branched configuration with one or more unsaturated carbon-carbon bonds that may occur in any stable point along the chain, such as ethenyl and propenyl. The language “C₂₋₂₀ alkenyl” includes branched and straight chain hydrocarbon groups with between 1 and 20 carbons and with one or more unsaturated carbon-carbon bonds. Moreover, the term “alkenyl” includes both “unsubstituted alkenyls” and “substituted alkenyls,” the latter of which refers to alkenyl moieties having substituents (e.g., F, Cl, Br, I, NO₂, CN, alkyl, aryl, hydroxyl, alkoxy, COCH₃ and the like) replacing a hydrogen on one or more carbons of the hydrocarbon backbone.

The term “aryl” includes 5-10 membered monocyclic, bicyclic, or tricyclic rings, wherein at least one ring, if more than one is present, is aromatic. The term “aryl” also includes “heteraryl” moieties in which one heteroatom (e.g., N, O or S) replaces one or more carbons in the monocyclic, bicyclic, or tricyclic ring. The term “aryl” also includes both “unsubstituted aryls” and “substituted aryls,” the latter of which refers to aryl moieties having substituents (e.g., F, Cl, Br, I, NO₂, CN, alkyl, hydroxyl, alkoxy, COCH₃ and the like) replacing a hydrogen on one or more carbons aromatic ring.

The term “hydroxyl” includes —OH.

The term “alkoxy” includes moieties in which an O is covalently bonded to a C₁₋₂₀ alkyl group, as defined above.

In some embodiments, the organopolysiloxane is vinyl terminated. In some embodiments, the organopolysiloxane is substantially vinyl terminated. The language “vinyl terminated organopolysiloxane” includes organopolysiloxanes that have at least one vinyl group at both terminal ends of the polymer. In certain embodiments, the language “vinyl terminated organopolysiloxane” includes organopolysiloxanes of formula II in which one or both of R^{2a} and R^{7a} are substituted with a C₂ alkyl moiety, for example, a vinyl moiety (e.g., —CH═CH₂). In a specific embodiment, a “vinyl terminated organopolysiloxane” includes organopolysiloxanes of formula II in which one or both of R^{2a} and R^{7a} are substituted with a C₂ alkyl moiety, for example, a vinyl moiety (e.g., —CH═CH₂), and R^{1a}, R^{3a}, R^{4a}, R^{5a}, R^{6a}, R^{8a}, R^{9a} and R^{10a} are independently selected from C₁₋₂₀ alkyl, for example, methyl.

In other embodiments, the organopolysiloxane is selected from: vinyl terminated polydimethylsiloxane; vinyl terminated diphenylsiloxane-dimethylsiloxane copolymers; vinyl terminated polyphenylmethylsiloxane, vinylphenylmethyl terminated vinylphenylsiloxane-phenylmethylsiloxane copolymer; vinyl terminated trifluoropropylmethylsiloxane-dimethylsiloxane copolymer; vinyl terminated diethylsiloxane-dimethylsiloxane copolymer; vinylmethylsiloxane-dimethylsiloxane copolymer, trimethylsiloxy terminated; vinylmethylsiloxane-dimethylsiloxane copolymers, silanol terminated; vinylmethylsiloxane-dimethylsiloxane copolymers, vinyl terminated; vinyl gums; vinylmethylsiloxane homopolymers; vinyl T-structure polymers; monovinyl terminated polydimethylsiloxanes; vinylmethylsiloxane terpolymers; vinylmethoxysilane homopolymers and combinations thereof.

In some embodiments, the organopolysiloxane is a high viscosity organopolysiloxane, a low viscosity organopolysiloxane or a combination thereof.

When the organopolysiloxane is a combination of high and low viscosity organopolysiloxanes, the combination of a high viscosity and a low viscosity vinyl organosiloxane provides a bimodal distribution of organosiloxane molecular weights. In at least one embodiment, the organopolysiloxane is a combination of high and low viscosity vinyl-terminal organopolysiloxanes providing a bimodal distribution of the vinyl-terminated organopolysiloxane. In one embodiment, the organopolysiloxane is a combination of formulas I, II, IIa, IIb, and IIc, for example, a combination of formula IIa, IIb and/or IIc, providing a bimodal distribution of the vinyl-terminated organopolysiloxane. In one embodiment, the organopolysiloxane is a combination of formula IIb and IIc. In one embodiment, the bimodal distribution of polymer molecular weight is represented by a ratio of the molecular weights (for example, the sum of s and t) of the high viscosity organopolysiloxanes to the low viscosity organopolysiloxane. In one embodiment, this ratio is from 2 to 3. In a specific embodiment, this ratio is 2.5.

The term “viscosity” refers to the measure of the resistance of a fluid which is being deformed by either shear stress or tensile stress. Viscosity may be reported as either dynamic viscosity, also absolute viscosity, (typical units Pa·s, Poise, P, cP) or kinematic viscosity (typical units cm²/s, Stokes, St, cSt), which is the dynamic viscosity divided by the density. Thus, if and when the density of a fluid is approximately 1, then the dynamic viscosity and the kinematic viscosity are equivalent. One of skill in the art would understand that the density of the fluid may vary with temperature or pressure, and as such would be able to adjust such measurements accordingly. One of skill in the art without undue experimentation would be able to determine how to measure the viscosity of a fluid, for example, using a viscometer or a rheometer. Representative methods include use of a capillary viscometer, rotational viscometer or rheometer to measure viscosity at an instrument specific strain. Specific methods for determining the viscosity of a fluid have been described in Example 8 of PCT/US2011/050003, published as WO2012/030984, the teachings of which have been incorporated herein by reference.

The language “high viscosity organopolysiloxane” includes organopolysiloxanes with a viscosity of between about 100,000 and about 500,000 cSt or cP at 25° C., for example, between about 110,000 and about 450,000 cSt or cP at 25° C., between about 120,000 and about 400,000 cSt or cP at 25° C., between about 125,000 and about 350,000 cSt or cP at 25° C., between about 130,000 and about 300,000 cSt or cP at 25° C., between about 135,000 and about 250,000 cSt or cP at 25° C., between about 140,000 and about 200,000 cSt or cP at 25° C., between about 145,000 and about 190,000 cSt or cP at 25° C., between about 150,000 and about 185,000 cSt or cP at 25° C., between about 155,000 and about 175,000 cSt or cP at 25° C., or between about 160,000 and about 170,000 cSt or cP at 25° C. In some embodiments, the viscosity of the high viscosity organopolysiloxane is between about 140,000 and about 200,000 cSt or cP at 25° C. In one embodiment, the high viscosity organopolysiloxane has a viscosity of about 165,000 cSt or cP at 25° C.

In one embodiment, the average molecular weight of the high viscosity organopolysiloxane is between about 100,000 and about 200,000 Da, for example, between about 115,000 and about 195,000 Da, between about 120,000 and about 190,000 Da, between about 125,000 and about 185,000 Da, between about 130,000 and about 180,000 Da, between about 135,000 and about 175,000 Da, between about 140,000 and about 170,000 Da, between about 145,000 and about 165,000 Da or between about 150,000 and about 160,000 Da. In one embodiment, the average molecular weight of the high viscosity organopolysiloxane is about 155,000 Da.

In some embodiments, the high viscosity organopolysiloxane is of formula II, in which R^{2a} and R^{7a} are C₂₋₂₀ alkenyl, for example, C₂ alkenyl (e.g., vinyl) and R^{1a}, R^{3a}, R^{4a}, R^{5a}, R^{6a}, R^{8a}, R^{9a} and R^{10a} are each C₁₋₂₀ alkyl, for example, C₁ alkyl (e.g., methyl). In some embodiments, the high viscosity organopolysiloxane is vinyl terminated. In other embodiments, the high viscosity organopolysiloxane is vinyl terminated polydimethylsiloxane.

In some embodiments, the vinyl terminated high viscosity organopolysiloxane has a weight percent of vinyl of between about 0.010 and about 0.100, for example, between about 0.015 and about 0.080, between about 0.020 and about 0.075, between about 0.025 and about 0.060, or between about 0.030 and about 0.050. In one embodiment, the high viscosity organopolysiloxane has a weight percent of vinyl of between about 0.030 and about 0.040.

In other embodiments, the high viscosity organopolysiloxane has a vinyl equivalent per kilogram of between about 0.0100 and about 0.0200, for example, between about 0.0110 and about 0.0190, between about 0.0115 and about 0.0180, between about 0.0120 and about 0.0170, between about 0.0125 and about 0.0165 or between about 0.013 and about 0.016.

In one embodiment, the high viscosity organopolysiloxane has on average at least two vinyl units per high viscosity organopolysiloxane. In one embodiment, the monomer unit including a vinyl moiety are spaced throughout the polymer. In one embodiment, the vinyl-containing monomer unit is spaced about 2000 monomer units away from another vinyl-containing monomer unit or a vinyl-containing terminal cap. For example, the vinyl units in the high viscosity organopolysiloxanes are separated by 1500, 1600, 1700, 1800, 1900, 2000, 2100, 2200, 2300, 2400, or 2500 monomer units.

In some embodiments, the high viscosity organopolysiloxane is selected from: vinyl terminated polydimethylsiloxane; vinyl terminated diphenylsiloxane-dimethylsiloxane copolymers; vinyl terminated polyphenylmethylsiloxane, vinylphenylmethyl terminated vinylphenylsiloxane-phenylmethylsiloxane copolymer; vinyl terminated trifluoropropylmethylsiloxane-dimethylsiloxane copolymer; vinyl terminated diethylsiloxane-dimethylsiloxane copolymer; vinylmethylsiloxane-dimethylsiloxane copolymer, trimethylsiloxy terminated; vinylmethylsiloxane-dimethylsiloxane copolymers, silanol terminated; vinylmethylsiloxane-dimethylsiloxane copolymers, vinyl terminated; vinyl gums; vinylmethylsiloxane homopolymers; vinyl T-structure polymers; monovinyl terminated polydimethylsiloxanes; vinylmethylsiloxane terpolymers; vinylmethoxysilane homopolymers and combinations thereof.

The language “low viscosity organopolysiloxane” includes organopolysiloxanes with a viscosity of between about 500 and about 50,000 cSt or cP at 25° C., for example, between about 1,000 and about 45,000 cSt or cP at 25° C., between about 1,500 and about 40,000 cSt or cP at 25° C., between about 2,000 and about 35,000 cSt or cP at 25° C., between about 2,500 and about 30,000 cSt or cP at 25° C., between about 3,000 and about 25,000 cSt or cP at 25° C., between about 3,500 and about 20,000 cSt or cP at 25° C., between about 4,000 and about 15,000 cSt or cP at 25° C., or between about 4,000 and about 12,000 cSt or cP at 25° C. In some embodiments, the low viscosity organopolysiloxane includes organopolysiloxanes with a viscosity of between about 100 and about 5,000 cSt or cP at 25° C., for example, between about 200 and about 4000 cSt or cP at 25° C., between about 300 and about 3000 cSt or cP at 25° C., between about 400 and about 2000 cSt or cP at 25° C. or between about 750 and about 1500 cSt or cP at 25° C. In one embodiment, the low viscosity organopolysiloxane has a viscosity of about 10,000 cSt or cP at 25° C. In some embodiments, the low viscosity organopolysiloxane has a viscosity of about 1000 cSt or cP at 25° C.

In some embodiments, the low viscosity organopolysiloxane has an average molecular weight of between about 20,000 and about 80,000 Da, for example, between about 50,000 and about 75,000 Da, between about 55,000 and about 70,000 Da, between about 60,000 and about 65,000 Da or between 62,000 and about 63,000 Da. In one embodiment, the low viscosity organopolysiloxane has an average molecular weight of about 62,700 Da. In one embodiment, the low viscosity organopolysiloxane has an average molecular weight of about 28,000 Da.

In some embodiments, the low viscosity organopolysiloxane is of formula II, in which R^{2a} and R^{7a} are C₂₋₂₀ alkenyl, for example, C₂ alkenyl (e.g., vinyl) and R^{1a}, R^{3a}, R^{4a}, R^{5a}, R^{6a}, R^{8a}, R^{9a} and R^{10a} are each C₁₋₂₀ alkyl, for example, C₁ alkyl (e.g., methyl). In some embodiments, the low viscosity organopolysiloxane is vinyl terminated. In some embodiments, the low viscosity organopolysiloxane is substantially vinyl terminated. In other embodiments, the low viscosity organopolysiloxane is vinyl terminated polydimethylsiloxane.

In some embodiments, the low viscosity organopolysiloxane has a weight percent of vinyl of between about 0.010 and about 0.30, for example, between about 0.020 and about 0.29, between about 0.030 and about 0.28, between about 0.040 and about 0.27, between about 0.050 and about 0.26, between about 0.060 between about 0.25, between about 0.070 and about 0.24, between about 0.080 and about 0.23, or between about 0.090 and about 0.22. In some embodiments, the low viscosity organopolysiloxane has a weight percent of vinyl of between about 0.18 and about 0.26.

In other embodiments, the low viscosity organopolysiloxane has a vinyl equivalent per kilogram of between about 0.010 and about 0.100, for example, between about 0.015 and about 0.090, between about 0.020 and about 0.080, between about 0.025 and about 0.070, between about 0.030 and about 0.060 or between about 0.040 and about 0.050. In some embodiments, the low viscosity organopolysiloxane has a vinyl equivalent per kilogram of between about 0.030 and about 0.040.

In other embodiments, the low viscosity organopolysiloxane has on average at least two vinyl units per low viscosity organpolysiloxane. In one embodiment, the monomer unit including a vinyl moiety are spaced throughout the polymer. In one embodiment, the vinyl-containing monomer unit is spaced about 850 monomer units away from another vinyl-containing monomer unit or a vinyl-containing terminal cap. For example, the vinyl units in the low viscosity organopolysiloxanes are separated by 450, 550, 650, 750, 800, 850, 950, 1050, 1150, 1250, or 1350 monomer units.

In some embodiments, the low viscosity organopolysiloxane is selected from: vinyl terminated polydimethylsiloxane; vinyl terminated diphenylsiloxane-dimethylsiloxane copolymers; vinyl terminated polyphenylmethylsiloxane, vinylphenylmethyl terminated vinylphenylsiloxane-phenylmethylsiloxane copolymer; vinyl terminated trifluoropropylmethylsiloxane-dimethylsiloxane copolymer; vinyl terminated diethylsiloxne-dimethylsiloxane copolymer; vinylmethylsiloxane-dimethylsiloxane copolymer, trimethylsiloxy terminated; vinylmethylsiloxane-dimethylsiloxane copolymers, silanol terminated; vinylmethylsiloxane-dimethylsiloxane copolymers, vinyl terminated; vinyl gums; vinylmethylsiloxane homopolymers; vinyl T-structure polymers; monovinyl terminated polydimethylsiloxanes; vinylmethylsiloxane terpolymers; vinylmethoxysilane homopolymers and combinations thereof.

In some embodiments, the organopolysiloxane is a compound of formula In):

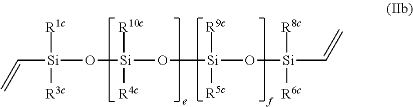

wherein R^{1c}, R^{3c}, R^{4c}, R^{5c}, R^{6c}, R^{8c}, R^{9c} and R^{10c} are each independently selected from hydrogen, C₁₋₂₀ alkyl, C₂₋₂₀ alkenyl, C₅₋₁₀ aryl, hydroxyl or C₁₋₂₀ alkoxyl and e and f are each independently an integer from between 10 and about 6000. In one embodiment, R^{1c}, R^{3c}, R^{4c}, R^{5c}, R^{6c}, R^{8c}, R^{9c} and R^{10c} are alkyl (e.g., C₁ alkyl, such as methyl). In some embodiments, the sum of e and f is an integer from about 1000 to about 8000; from about 1300 to about 2700; from about 1500 to about 2700; from about 1600 to about 2600; from about 1600 to about 2500; from about 1700 to about 2500; from about 1800 to about 2400; from about 1800 to about 2300; from about 1900 to about 2300; from about 2000 to about 2200; from about 2050 to about 2150; from about 2100.

In some embodiments, the organopolysiloxane is a compound of formula IIc:

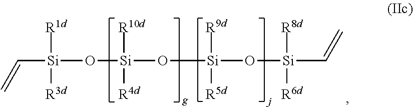

wherein R^{1d}, R^{3d}, R^{4d}, R^{5d}, R^{6d}, R^{8d}, R^{9d} and R^{10d} are each independently selected from hydrogen, C₁₋₂₀ alkyl, C₂₋₂₀ alkenyl, C₅₋₁₀ aryl, hydroxyl or C₁₋₂₀ alkoxyl and g and j are each independently an integer from between 10 and about 6000. In one embodiment, R^{1a}, R^{3d}, R^{4d}, R^{5d}, R^{6d}, R^{8d}, R^{9d} and R^{10d} are alkyl (e.g., C₁ alkyl, such as methyl). In some embodiments, the sum of g and j is an integer from about 200 to about 1100; from about 600 to about 1100; from about 700 to about 1000; from about 800 to about 900; from about 825 to about 875; from about 850; from about 200 to about 800; from about 225 to about 700; from about 250 to about 600; from about 275 to about 500; from about 300 to about 400; from about 350 to about 400; from about 375. In some embodiments, the sum of g and j is an integer from about 850.

In some embodiments, the reactive constituent comprises at least one hydride functionalized polysiloxane. The language “hydride functionalized polysiloxane” includes compounds of formula III:

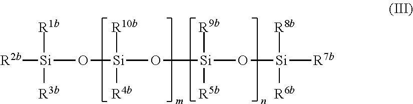

wherein R^{1b}, R^{2b}, R^{3b}, R^{4b}, R^{5b}, R^{6b}, R^{7b}, R^{8b}, R^{9b} and R^{10b} are each independently selected from hydrogen, C₁₋₂₀ alkyl, C₂₋₂₀ alkenyl, C₅₋₁₀ aryl, hydroxyl or C₁₋₂₀ alkoxy and m and n are each independently an integer from between 10 and about 6000, provided that at least one of R^{1b}, R^{2b}, R^{3b}, R^{4b}, R^{5b}, R^{6b}, R^{7b}, R^{8b}, R^{9b} and R^{10b} is hydrogen. In some embodiments, at least one of R^{1b}, R^{2b}, R^{3b}, R^{4b}, R^{5b}, R^{6b}, R^{7b}, R^{8b}, R^{9b} and R^{10b} is hydrogen and the remainder are C₁₋₂₀ alkyl. In some embodiments, at least two of R^{1b}, R^{2b}, R^{3b}, R^{4b}, R^{5b}, R^{6b}, R^{7b}, R^{8b}, R^{9b} and R^{10b} are hydrogen (e.g., two Si—H units per functionalized hydride polysiloxane molecule). In other embodiments, at least three of R^{1b}, R^{2b}, R^{3b}, R^{4b}, R^{5b}, R^{6b}, R^{7b}, R^{8b}, R^{9b} and R^{10b} are hydrogen (e.g., three Si—H units per functionalized hydride polysiloxane molecule). In some embodiments, at least two of R^{1b}, R^{2b}, R^{3b}, R^{4b}, R^{5b}, R^{6b}, R^{7b}, R^{8b}, R^{9b} and R^{10b} are hydrogen (e.g., two Si—H units per functionalized hydride polysiloxane molecule) and the remainder are C₁₋₂₀ alkyl. In other embodiments, at least three of R^{1b}, R^{2b}, R^{3b}, R^{4b}, R^{5b}, R^{6b}, R^{7b}, R^{8b}, R^{9b} and R^{10b} are hydrogen (e.g., three Si—H units per functionalized hydride polysiloxane molecule) and the remainder are C₁₋₂₀ alkyl. In some embodiments, at least two of R^{4b}, R^{5b}, R^{9b} and R^{10b} are hydrogen (e.g., two Si—H units per functionalized hydride polysiloxane molecule) and the remainder are C₁₋₂₀ alkyl. In other embodiments, at least three of R^{4b}, R^{5b}, R^{9b} and R^{10b} are hydrogen (e.g., three Si—H units per functionalized hydride polysiloxane molecule) and the remainder are C₁₋₂₀ alkyl.

In one embodiment, at least greater than two monomer units of formula III include a —Si—H unit (e.g. one or more of R^{4b}, R^{5b}, R^{9b} and R^{10b} is hydrogen). For example, on average 2 to 15 monomer units of formula III include a Si—H unit. In one embodiment, at least two monomer units of formula III include a —Si—H unit (e.g. one or more of R^{4b}, R^{5b}, R^{9b} and R^{10b} is hydrogen). In one embodiment, at least three monomer units of formula III include a —Si—H unit (e.g. one or more of R^{4b}, R^{5b}, R^{9b} and R^{10b} is hydrogen). In one embodiment, at least four monomer units of formula III include a —Si—H unit (e.g. one or more of R^{4b}, R^{5b}, R^{9b} and R^{10b} is hydrogen). In one embodiment, at least five monomer units of formula III include a —Si—H unit (e.g. one or more of R^{4b}, R^{5b}, R^{9b} and R^{10b} is hydrogen). In one embodiment, at least six monomer units of formula III include a —Si—H unit (e.g. one or more of R^{4b}, R^{5b}, R^{9b} and R^{10b} is hydrogen). In one embodiment, at least seven monomer units of formula III include a —Si—H unit (e.g. one or more of R^{4b}, R^{5b}, R^{9b} and R^{10b} is hydrogen). In one embodiment, at least eight monomer units of formula III include a —Si—H unit (e.g. one or more of R^{4b}, R^{5b}, R^{9b} and R^{10b} is hydrogen). In a specific embodiment, the non Si—H positions may include a Si-(alkyl) or Si-(vinyl) unit. In a specific embodiment, the non-Si—H positions are Si—CH₃. In a specific embodiment, the Si—H positions are not present in the terminal caps. In one embodiment, the Si—H units in the hydride-functionalized organopolysiloxanes are separated by 5, 10, 15, 20, 25, 30, 35, 40, 45, 50, 60, 65, 70, 75, 80, 85, 90, 100, 125, 150, or 200 monomer units.

In one aspect of any one of the above embodiments, the sum of m and n is an integer from about 10 to about 1300; from about 10 to about 1100; from about 10 to about 600; from about 15 to about 500; from about 15 to about 400; from about 20 to about 300; from about 20 to about 200; from about 25 to about 100; from about 25 to about 75; from about 30 to about 50; from about 40.

In some embodiments, the hydride functionalized polysiloxane includes Si—H units only at the terminal caps of the polymer. In some embodiments, the polysiloxane include Si—H units only in the monomer units, but not at the terminal caps of the polymer. In other embodiments, the polysiloxane includes Si—H units at both the terminal cap or in the monomer unit of the polymer. In one embodiment, the polysiloxane includes two to twelve Si—H units on average located either at the terminal cap, or within the monomer unit, or a combination thereof. In one embodiment, the polysiloxane includes four to fifteen Si—H units on average located either at the terminal cap, or within the monomer unit, or a combination thereof. In one embodiment, the polysiloxane includes eight Si—H units on average located either at the terminal cap, or within the monomer unit, or a combination thereof. In one embodiment, the polysiloxane includes two to twelve Si—H units on average located within the monomer unit, and not at the terminal caps. In one embodiment, the polysiloxane includes four to fifteen Si—H units on average located within the monomer unit, and not at the terminal caps. In one embodiment, the polysiloxane includes eight Si—H units on average located within the monomer unit, and not at the terminal caps.

In some embodiments, the hydride functionalized polysiloxane has a viscosity of between about 5 and about 11,000 cSt or cP at 25° C., for example, between about 10 and about 10,000 cSt or cP at 25° C., between about 15 and about 5,000 cSt or cP at 25° C., between about 20 and about 1,000 cSt or cP at 25° C., between about 25 and about 500 cSt or cP at 25° C., between about 30 and about 100 cSt or cP at 25° C., and between about 40 and about 50 cSt or cP at 25° C. In one embodiment, the hydride functionalized polysiloxane has a viscosity of about 45 cSt or cP at 25° C.

In some embodiments, the hydride functionalized polysiloxane has an average molecular weight of between about 900 and about 60,000 Da, for example, between about 1000 and about 50,000 Da, between about 1200 and about 25,000 Da, between about 1400 and about 20,000 Da, between about 1600 and about 15,000 Da, between about 1800 and about 10,000 Da, between about 2000 and about 5000 Da, between about 2200 and about 4000 Da, and between 2300 and about 2500 Da. In one embodiment, the average molecular weight of the hydride functionalized polysiloxane is about 2400 Da.

In some embodiments, the hydride functionalized polysiloxane has a percent SiH content of between about 3 and about 45%, for example, between about 5 and about 40%, between about 10 and about 35%, between about 20 and about 30%, or between about 26 and 27%. In some embodiments, the hydride functionalized polysiloxane has a percent SiH content of about 26%.

In some embodiments, the hydride functionalized polysiloxane has an SiH content of between about 0.500 mmol/g and about 10.00 mmol/g, for example, between about 1.00 mmol/g and about 9.00 mmol/g, between about 2.00 and about 8.00 mmol/g, between about 3.00 mmol/g and about 7.00 mmol/g, and about 4.00 mmol/g and about 6.00 mmol/g. In one embodiment, the hydride functionalized polysiloxane has an SiH content of between about 4.00 and about 5.00 mmol/g, for example, 4.35 mmol/g.

In other embodiments, the hydride functionalized polysiloxane is alkyl terminated. In other embodiments, the hydride functionalized polysiloxane is substantially alkyl terminated. The language “alkyl terminated” includes hydride functionalized polysiloxanes of formula III in which one or both of R^{2b} and R^{7b} are C₁₋₂₀ alkyl. In some embodiments, “alkyl terminated” includes hydride functionalized polysiloxanes of formula III in which one, two, three, four, five or six of R^{1b}, R^{2b}, R^{3b}, R^{6b}, R^{7b} and R^{8b} are C₁₋₂₀ alkyl. In one embodiment, R^{1b}, R^{2b}, R^{3b}, R^{4b}, R^{5b}, R^{6b}, R^{7b}, R^{8b} and R^{10b} are each C₁₋₂₀ alkyl, for example, C₁ alkyl (e.g., methyl) and R^{9b} is hydrogen. In one embodiment, R^{1b}, R^{2b}, R^{3b}, R^{4b}, R^{5b}, R^{6b}, R^{7b}, R^{8b} and R^{9b} are each C₁₋₂₀ alkyl, for example, C₁ alkyl (e.g., methyl) and R^{10b} is hydrogen.

In some embodiments, the hydride functionalized polysiloxane is selected from the group consisting of hydride terminated polydimethylsiloxane; polyphenyl-(dimethylhydrosiloxy)siloxane, hydride terminated; methylhydrosiloxane-phenylmethylsiloxane copolymer, hydride terminated; methylhydrosiloxane-dimethylsiloxane copolymers, trimethylsiloxy terminated; polymethylhydrosiloxanes, trimethylsiloxy terminated; polyethylhydrosiloxane, triethylsiloxane, methylhydrosiloxane-phenyloctylmethylsiloxane copolymer; methylhydrosiloxane-phenyloctylmethylsiloxane terpolymer and combinations thereof.

In some embodiments, the reactive constituent comprises combinations of polymers of formulas I, II, IIa, IIb, IIc, IId, and/or III. In a specific embodiment, the reactive constituent comprises a combination of polymers of formulas IIa, IIb, IIc and/or III. In a specific embodiment, the reactive constituent comprises a combination of polymers of formulas IIb, IIc and III.

In some embodiments, the reactive constituent comprises combinations of high molecular weight vinyl organopolysiloxanes, low molecular weight vinyl organopolysiloxanes, and/or hydride-functionalized organopolysiloxanes. In one embodiment, each of the high and low molecular weight organopolysiloxanes includes on average at least two vinyl moieties per polymer. In a specific embodiment, each vinyl organopolysiloxane includes exactly two vinyl moieties on average. In one embodiment, the ratio of the high molecular organopolysiloxane to the low molecular weight organopolysiloxane is 2 to 3, for example 2, 2.5 or 3. The ratio may be selected in order to adjust the chemical and physical properties of the film in order to suit a specific method or part of the body. In one embodiment, the hydride-functionalized organopolymer includes on average greater than two Si—H units in the polymer. In a specific embodiment, there are 8 Si—H units on average per hydride-functionalized organopolysiloxane.

In some embodiments, the reactive constituent comprises combinations of high molecular weight hydride-functionalized organopolysiloxanes, low molecular weight hydride functionalized organopolysiloxanes, and/or vinyl organopolysiloxanes. In one embodiment, each of the high and low molecular weight organopolysiloxanes include on average at least two Si—H units per polymer. In a specific embodiment, each hydride-functionalized organopolysiloxane includes exactly two Si—H moieties. In one embodiment, the ratio of the high molecular organopolysiloxane to the low molecular weight organopolysiloxane is 2 to 3, for example 2, 2.5 or 3. The ratio may be selected in order to adjust the chemical and physical properties of the film in order to suit a specific method or part of the body. In one embodiment, the vinyl organopolymer includes on average greater than at least two vinyl units in the polymer. In a specific embodiment, there are 8 vinyl units on average per vinyl organopolysiloxane.

The language “reinforcing constituent” includes one or more constituents of the reactive reinforcing component that provide the required physical properties of the film that results from the in situ reaction between the reactive reinforcing component and the cross-linking component. Such physical properties include, for example, mechanical elements (e.g., elasticity, durability, fracture strain, tensile strength, etc. . . . ), biocompatibility (e.g., selective breathability, adhesion, etc. . . . ), optical effects (e.g., reflectance, color, etc. . . . ) and surface modulation (e.g., texture, chemistry, etc. . . . ). Examples of reinforcing constituents include clays, (e.g., Al₂O₃, SiO₂), chalk, talc, calcite (e.g., CaCO₃), mica, barium sulfate, zirconium dioxide, zinc sulfide, zinc oxide, titanium dioxide, aluminum oxide, silica aluminates, calcium silicates, or optionally surface treated silica (e.g., fumed silica, hydrated silica or anhydrous silica). In some embodiments, reinforcing constituent is silica, for example, surface treated silica, such as silica treated with hexamethyldisilazane. In some embodiments, fumed silica has been surface treated with hexamethyldilazane.

In some embodiments, the reinforcing constituent has a surface area of between about 100 and about 300 m²/g, for example, between about 110 and about 250 m²/g, between about 120 and about 225 m²/g, between about 130 and about 200 m²/g, between about 135 and about 185 m²/g, between about 160 and about 170 m²/g, and between about 164 and about 166 m²/g. In one embodiment, the reinforcing constituent has a surface area of about 160±25 m²/g.

In some embodiments, the reinforcing constituent has an average particle size of between about 1 and about 20 μm.

In some embodiments, the reinforcing constituent is compounded with the low viscosity and/or the high viscosity organopolysiloxane.

In some embodiments, reactive constituent and reinforcing constituent comprise between about 20 and about 90% of the reactive reinforcing component, for example, between about 40% and about 60% of the reactive reinforcing component. In some embodiments, the reactive constituent and reinforcing constituent comprise between about 45.0 and about 61.0% of the reactive reinforcing component, for example, about 45.0%, about 45.5%, about 46.0%, about 46.5%, about 47.0%, about 47.5%, about 48.5%, about 49.0%, about 49.5%, about 50.0%, about 50.5%, about 51.0%, about 51.5%, about 52.0%, about 52.5%, about 53.0%, about 53.5%, about 54.0%, about 54.5%, about 55.0%, about 55.5%, about 56.0%, about 56.5%, about 57.0%, about 58.0%, about 58.5%, about 59.0%, about 59.5%, about 60.0%, or about 60.5%. In some embodiments, the reactive constituent and the reinforcing constituent comprise about 45% of the reactive reinforcing component. In one embodiment, the reactive constituent and reinforcing constituent comprise about 48.0% of the reactive reinforcing component. In some embodiments, the reactive constituent and the reinforcing constituent comprise about 50.0% of the reactive reinforcing component. In another embodiment, the reactive constituent and reinforcing constituent comprise about 51.0% of the reactive reinforcing component. In some embodiments, the reactive constituent and the reinforcing constituent comprise about 51.5% of the reactive reinforcing component. In another embodiment, the reactive constituent and reinforcing constituent comprise about 54.5% of the reactive reinforcing component. In another embodiment, the reactive constituent and reinforcing constituent comprise about 55.0% of the reactive reinforcing component. In some embodiments, the reactive constituent and the reinforcing constituent comprise about 59.5% of the reactive reinforcing component. In another embodiment, the reactive constituent and reinforcing constituent comprise about 60.5% of the reactive reinforcing component. In some embodiments, the reactive constituent and reinforcing constituent comprise between about 30.0 and about 40.0% of the reactive reinforcing component, for example, about 30.0%, about 30.5%, about 31.0%, about 31.5%, about 32.0%, about 32.5%, about 33.0, about 33.5%, about 34.0%, about 34.5%, about 35.0%, about 35.5%, about 36.0%, about 36.5%, about 37.0%, about 37.5%, about 38.0%, about 38.5%, about 39.0%, about 39.5%, about 40.0%. In some embodiments, the reactive constituent and reinforcing constituent comprise between about 33.0 and about 40.0% of the reactive reinforcing component

In one embodiment, the reinforcing constituent comprises between about 8.0 and about 13.0% of the reactive reinforcing component, for example, about 8.5%, about 9.0%, about 9.5%, about 10.0%, about 10.5%, about 11.0%, about 11.5%, about 12.0% or about 12.5%. In some embodiments, the reinforcing constituent comprises about 8.5% of the reactive reinforcing component. In one embodiment, the reinforcing constituent comprises about 9.0% of the reactive reinforcing component. In another embodiment, the reinforcing constituent comprises about 9.5% of the reactive reinforcing component. In some embodiments, the reinforcing constituent comprises about 10.0% of the reactive reinforcing component. In some embodiments, the reinforcing constituent comprises about 10.5% of the reactive reinforcing component. In another embodiment, the reinforcing constituent comprises about 11.0% of the reactive reinforcing component. In another embodiment, the reinforcing constituent comprises about 12.0% of the reactive reinforcing component. In another embodiment, the reinforcing constituent comprises about 13.0% of the reactive reinforcing component.

In another embodiment, the reactive constituent comprises between about 30.0 and about 60.0% of the reactive reinforcing component, for example, about 30.5%, about 31.0%, about 32.0%, about 33.0%, about 34%, about 35.0%, about 36.0%, about 37.0%, about 38.0%, about 39.0%, about 40.0%, about 41.0%, about 42.0%, about 43.0%, about 44.0%, about 45.0%, about 46.0%, about 47.0%, about 48.0%, about 49.0%, about 50.0%, about 51.0%, about 52.0%, about 53.0%, about 54.0%, about 55.0%, about 56.0%, about 57.0%, about 58.0% or about 59.0%.

In some embodiments, the reactive reinforcing component has a viscosity of between about 5,000 and 1,000,000 cSt or cP at 25° C. In some embodiments, the reactive reinforcing component has a viscosity of between about 10,000 and 10,000,000 cSt or cP at 25° C., for example, about 10,000,000, about 9,000,000, about 8,000,000, about 7,000,000, about 6,000,000, about 5,000,000, about 4,000,000, about 3,000,000 or about 2,000,000, about 1,000,000, about 900,000, about 800,000, about 700,000, about 600,000, about 500,000, about 400,000, about 300,000, about 200,000, about 100,000, about 90,000, about 80,000, about 70,000, about 60,000, about 50,000, about 40,000, about 30,000, about 20,000, about 10,000 cSt. In one embodiment, the reactive reinforcing component has a viscosity of about 1,000,000 cSt.

In some embodiments, the reactive reinforcing component has a vinyl to functional hydride (e.g., —CH═CH₂ of the one or more organopolysiloxanes to Si—H of the hydride functionalized polysiloxane) ratio of between about 1:10 and about 1:100, for example, between about 1:15 and about 1:90, between about 1:20 and about 1:80, between about 1:25 and about 1:70, between about 1:30 and about 1:60, between about 1:35 and about 1:50. In one embodiment, the reactive reinforcing component has a vinyl to functional hydride ratio of about 1:40. In another embodiment, the reactive reinforcing component has a vinyl to functional hydride ratio of about 1:20. In some embodiments, the reactive reinforcing component has a vinyl to functional hydride ratio of about 1:15.

The language “cross-linking component” includes a component that, when applied to the reactive reinforcing component, catalyzes the in situ formation of the film. Similarly, “cross-linking component” includes a component that, when applied to the reactive reinforcing component, facilitates in situ formation of the film

The term “catalyzes the in situ formation of a film” or “facilitates in situ formation of the film” includes causing a reaction to occur between the reactive constituents of the reactive reinforcing component, such that a film is formed on the skin. Without being bound by theory, the cross-linking component induces a reaction between the one or more organopolysiloxanes and the hydride functionalized polysiloxane of the reactive reinforcing component causing the condensation of these constituents, such that a film is formed upon the skin.

The language “the film is formed” and “film formation” includes the results of the polymerization reaction that occurs upon the interaction of the color corrective composition and reactive reinforcing component and the cross-linking component. Without being bound by theory, film formation is characterized by a phase transition from the viscous sol state of a mixture to that of a continuous interconnected polymer state of film.

A technician could determine when the film is formed on the skin by using routine methods. For example, rheological measurements using small amplitude oscillatory shear can determine the continuous evolution of the viscoelastic properties, such as elastic modulus (G′), the viscous modulus (G″) and the loss of tangent (tan δ) of the reacting mixture continuously through the film formation process. In some embodiments, the rheometer can be used to determine the cross over time between G′ and G″ and the time when tan δ becomes frequency independent, which is a measure of film formation. In some embodiments, the film is formed within at least about five minutes, for example, within about one minute, about two minutes, about three minutes or about four minutes. In some embodiments, the film is formed within at least about 10 seconds and about 3 minutes.

In some embodiments, the film has a Young's Modulus (e.g., tensile strength) of between about 0.01 and about 1 MPa.

In some embodiments, the fracture strain of the film has a fracture strain of at least about 150%.

In some embodiments, the film has a leather adhesive force of greater than about 20 N/mm, for example, greater than about 25 N/mm, greater than about 30 N/mm, greater than about 35 N/mm, greater than about 40 N/mm, greater than about 45 N/mm, greater than about 50 N/mm, greater than about 55 N/mm, greater than about 60 N/mm, greater than about 65 N/mm, greater than about 70 N/mm, greater than about 75 N/mm, or greater than about 80 N/mm. In one embodiment, the leather adhesive force is between about 50 and about 80 N/mm.

In some embodiments, the film has a hysteresis of less than about 10% for example, least than about 9%, less than about 8%, less than about 7%, less than about 6%, less than about 5%, less than about 4%, less than about 3%, less than about 2%, less than 1% or about 0%.

In some embodiments, the film is between about 10 μm and about 1500 μm thick, for example, between about 50 μm and about 500 μm thick. In some embodiments, the film is less than about 100 μm thick. The film thickness may be measured by methods known to one of skill in the art, for example, by the combination of calipers and a calibrated microscope. The thickness of the film may also be digitally measured from a micrograph of the film cross-section. The microscope calibration allows for the conversion of measured pixelar distance into metric distance units.

In some embodiments, the film shrinks by less than between about 1 and 30%, for example, between about 1 to about 15%. The amount of shrinking may be determined by methods known to one of skill in the art, for example, by the Croll method (Croll, S. G. J. Coatings Tech. 52 (1980) 35, the teachings of which are incorporated herein by reference). In this method the film is used to coat one side of a thin flexible substrate. The amount of curve developed in the substrate due to the shrinking of the coating is used to calculate the magnitude of shrinking of the coating (Francis et al., J Mater Sci 2002; 37:4717-31, the teachings of which are incorporated herein by reference.)

In some embodiments, the film is physiologically stable. The language “physiologically stable” includes the durability of the film upon exposure to normal skin conditions, for example, humidity, tears, sweat or sebum. The physiological stability may be determined by methods typically used by one of ordinary skill in the art, such as an uptake test, which measures the change in weight of the film after exposure to a physiological factor. For example, the uptake test may employ a formulation of simulated sweat (e.g., 1× phosphate buffered saline solution) or simulated sebum (e.g., 25% wax monoesters, 41% triglycerides, 16% free fatty acids and 12% squalene). In some embodiments, the weight of the film increases by less than about 10%, for example, less than about 9%, less than about 8%, less than about 7%, less than about 6%, less than about 5%, less than 4%, less than 3%, less than 2%, less than 1% or exhibits no increase upon exposure to humidity, tears, sweat or sebum.

In some embodiments, the film as maintained on the skin for about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours or about 24 hours.

In some embodiments, the cross-linking component comprises a metal catalyst, for example, a platinum catalyst, a rhodium catalyst or a tin catalyst. Examples of platinum catalysts include, for example, platinum carbonyl cyclovinylmethylsiloxane complexes, platinum divinyltetramethyldisiloxane complexes, platinum cyclovinylmethylsiloxane complexes, platinum octanaldehyde/octanol complexes and combinations thereof. An example of a rhodium catalyst includes Tris(dibutylsulfide) Rhodium trichloride. Examples of tin catalysts include tin II octoate, Tin II neodecanoate, dibutyltin diisooctylmaleate, Di-n-butylbis(2,4 pentanedionate)tin, di-n-butylbutoxychlorotin, dibutyltin dilaurate, dimethyltin dineodecanoate, dimethylhydroxy(oleate)tin and tin II oleate.

In some embodiments, the cross-linking component further comprises a vinyl terminated organopolysiloxane (e.g., a compound of Formula I, II IIa, IIb or IIc). In some embodiments, the amount of vinyl-terminated polysiloxane is a stabilizing amount of vinyl-terminated polysiloxane. The language “stabilizing amount” includes an amount that prevents the degradation of the catalyst and/or the crosslinking component and/or the film. In some embodiments, the stabilizing amount of vinyl-terminated polysiloxane is less than about 50%, less than about 40%, less than about 30%, less than about 20%, less than about 10%, less than about 5% or less than about 2%. In some embodiments, the stabilizing amount of vinyl-terminated polysiloxane is about 1%.

In some embodiments, the cross-linking component has a viscosity of between about 1,000 and about 50,000 cSt or cP at 25° C.

In some embodiments, the catalyst is added as a solution and the solution comprises between about 1.0 and about 5.0% of the cross-linking component, for example, about 1.5%, about 2.0%, about 2.5%, about 3.0%, about 3.5%, about 4.0% or about 4.5%. In one embodiment, the catalyst is about 2.0% of the cross-linking component.

In some embodiments, the catalyst comprises between about 0.005 and about 0.04% of the cross-linking component, for example, about 0.005%, about 0.010%, about 0.015%, about 0.020%, about 0.025%, about 0.030% or about 0.035% or about 0.040%. In one embodiment, the catalyst is about 0.02% of the cross-linking component.

In some embodiments, the catalyst is present in the cross-linking component in an amount of between about 100 ppm and about 500 ppm.

In some embodiments, the film has the appearance of natural skin upon application to the skin. The language “appearance of natural skin” includes the perception that the film, when applied to the skin, has the look, feel and texture of real skin and that the film treated skin has the physical properties (e.g., the elasticity and stiffness) of real (e.g., live) skin. A trained observer and/or a technician would be able to determine whether the film upon application to the body has the appearance of natural skin. For example, a trained observer would be able to determine whether the film, upon application to the body, appears excessively shiny, or whether the film appears not to move with the underlying musculature of the skin by, for example, breaking, buckling or deforming, in response to natural skin motion.

A technician would be able to determine whether the film has the appearance of natural skin upon application to the body. For example, the elasticity and stiffness of skin, with or without the film applied to it, can be assessed by a wide variety of methods (Agache et al., Arch. Dermatol. Rev., 269 (1980) 221), the teachings of which are incorporated herein by reference. For example, the DermaLab suction cup instrument provides one common method to assess the mechanical properties of skin, and has previously shown younger skin to be less stiff and more elastic than aged skin (Grahame et al. Clinical Science 39 (1970) 223-238, the teachings of which are incorporated herein by reference). With this method, the stiffness of the skin is indicated by the Young's Modulus, a measure calculated by the instrument based on the pressure required to suck skin up a predetermined distance.

In some embodiments, the Young's Modulus of the skin treated with the film is reduced by between about 5% to about 70%, for example, between about 30% and about 60%, or between about 40% and about 50% compared to untreated skin. In some embodiments, the Young's Modulus of skin treated with the film is reduced by between about 5% and about 25% compared to untreated skin.

The elasticity of the skin is determined by the skin retraction time. The retraction time is obtained by measuring the time it takes for the skin to drop a predetermined distance towards its natural position, after the suction pressure is removed. In some embodiments, the retraction time of skin treated with the film is decreased by between about 5% and about 75%, for example, between about 30% and about 60%, or about 50% and about 65% when compared to untreated skin. In some embodiments, the retraction time of skin treated the film is decreased by between about 5% and about 10% compared to untreated skin. In some embodiments, the retraction time of the skin treated with the film approaches the retraction time of the film alone.

In some embodiments, the film, upon application to the skin, has the appearance and physical properties of youthful, unblemished natural skin. The language “youthful skin” includes skin that has mild or no damage, as measured by the Griffith's score. The Griffith's score (GS), as shown below, is a quantitative measurement of the amount of skin damage subject has.

A. 0-1: No damage

B. 2-3: Mild damage

C. 4-5: Moderate damage

D. 6-7: Moderate to severe damage

E. 8-9: Severe damage

In some embodiments, youthful skin includes skin that has a Griffith's score of between about 0 and about 3.

In some embodiments, the subject's skin has a negative change in Griffith's score (ΔGS) of about 1, about 2, about 3, about 4, about 5, about 6, about 7 or about 8 after application of the film. In some embodiments, the subject's skin has a ΔGS of between about −0.5 and about −3.0 upon application of the film. In one embodiment, the subject's skin has a ΔGS between about −1 and about −1.5, between about −1.2 and about −1.3 (e.g., about −1.25) upon application of the film. In another embodiment, the subject's skin has a ΔGS of between about −2.0 and about −3.0, for example, between about −2.0 and about −2.5, or between about −2.1 and about −2.2 (e.g., about −2.15) upon application of the film.

In other embodiments, the film, upon application to the skin, provides stiffness and elasticity such that the skin treated with the film appears substantially more similar to youthful skin than untreated skin. The term “elasticity” includes the skin's tendency to return to its original shape once it's been deformed. The language “elasticity substantially similar to youthful skin” includes the ability of the skin to return to its original shape once it's been deformed in a manner similar to that of young skin. The term “stiffness” includes the skin's resistance to deformation. The language “stiffness substantially similar to youthful skin” includes the ability of the skin to resist deformation in a manner similar to that of young skin. A technician would also be able to determine whether the film, upon application to the body, has the aforementioned physical properties of youthful, unblemished, natural skin by the techniques described above (e.g., using the Dermalab suction cup instrument).

In some embodiments, the subject and/or observers of the subject perceive an age reduction upon application of the film. In some embodiments, the perceived age reduction is about 1 year, about 2 years, about 3 years, about 4 years, about 5 years, about 6 years, about 7 years, about 8 years, about 9 years, about 10 years, about 11 years, about 12 years, about 13 years, about 14 years or about 15 years less than the subject's actual age. In some embodiments, the perceived age reduction is about 7.5 years less than the subject's actual age upon application of the film. In other embodiments, the perceived age reduction is about 8.5 years less than the subject's actual age upon application of the film.

### Example 1

### Formulations: Color Corrective Composition

Components of the formulations are commercially available. The following table provides the generic name for any trade name used throughout this application.

| | International Nomenclature Cosmetic |
|---|---|
| Tradename | Ingredient (INCI) name |
| Aerogel VM2270 | Silica Silylate |
| Aerosil 8200 ™ or Aerosil | Fumed silica modified with hexamethyldisilazane |
| R8200 ™ | |
| Andisil C1000 ™ | Silicon dioxide + Dimethylpolysiloxane |
| Andisil C1300 ™ | Silicon dioxide + Dimethylpolysiloxane |
| Andisil CE-4 ™ | Vinyl Dimethicone |
| Andisil MV 2,000 ™ or | Vinyl Dimethicone |
| MV2000 | |
| Andisil VS 1,000 ™ | Vinyl Dimethicone |
| Andisil VS 10,000 ™ or VS | Vinyl Dimethicone |
| 10,000 | |
| Andisil VS 165,000 ™ or | Vinyl Dimethicone |
| Andisil VS165K or VS 165,000 | |
| Andisil VS 20,000 ™ | Vinyl Dimethicone |
| Andisil VS 250 ™ | Vinyl Dimethicone |
| Andisil VS 500 ™ or VS500 | Vinyl Dimethicone |
| Andisil VS 65,000 ™ or | Vinyl Dimethicone |
| VS65,000 | |
| Andisil XL-11 ™ or XL-11 | Hydrogen Dimethicone, SiH Functional |
| Andisil XL-1B ™or XL-1B | Hydrogen Dimethicone, SiH Functional |
| Aquadispersable Rutile | Titanium dioxide |
| Titanium Dioxide ™ | |
| Barium Sulfate HL | Barium Sulfate |
| Beaver UV/Fluorescent | AROMATIC HETEROCYCLE |
| Pigment | |
| Cabosperse 1030K | CAB-O-SPERSE ® 1030K is an aqueous |
| | dispersion of CAB-O-SIL ® L-90, a very low |
| | surface area, fumed silica. It is electrostatically |
| | stabilized with Potassium Hydroxide and has an |
| | alkaline pH. |
| Carbopol Ultrez 21 | Acrylates/C10-30 Alkyl Acrylate Crosspolymer |
| Cetiol OE | Dicapryl Ether |
| Chronosphere Optical Brite or | Silica and polyurethane-40/silica and |
| Chronosphere Opticals/Opticals | polyurethane-40 and green 5 |
| Brite | |
| Covacryl MV60 | Sodium Polyacrylate |
| cremaphor EL | PEG-35 Castor Oil |
| Crodamol STS | PPG 3 Benzyl Ether Myristate |
| DC 200 Fluid (1 cSt) | Dimethicone |
| DC 2-1184 fluid (DOW | Trisiloxane (and) Dimethicone |
| CORNING ® 2-1184 FLUID) | |
| DC 556 | Phenyl Trimethicone |
| DMFS CS | dimethicone |
| DMS-V41 | Poly(Dimethylsiloxane), Vinyl Terminated |
| Dow 245 Fluid (Dow | Cyclopentasiloxane |
| CORNING 245 Fluid) | |
| Dow 246 Fluid (Dow | Cyclohexasiloxane |
| CORNING 246 Fluid) | |
| Dow 9011 Elastomer Blend | Cyclopentasiloxane (and) PEG-12 Dimethicone |
| (Dow Corning 9011 Elastomer | Crosspolymer |
| Blend) | |
| Dow Corning 9011 Silicone | Cyclopentasiloxane (and) PEG-12 Dimethicone |
| Elastomer Blend ™ or Dow | Crosspolymer |
| Elastomer Blend 9011 | |
| Dow 9045 Elastomer Blend or | Cyclopentasiloxane (and) Dimethicone |
| Dow Corning 9045 Silicone | Crosspolymer |
| Elastomer Blend ™ | |
| Dow Corning 200 Fluid 0.65 | Hexamethyldisiloxane |
| cSt ™ | |
| Dow Corning 245 Fluid ™ | Decamethylcyclopentasiloxane |
| Dow Corning 5329 | PEG-12 Dimethicone |
| Dow Elastomer Blend 9041 or | Dimethicone (and) Dimethicone Crosspolymer |
| DOW CORNING ® 9041 | |
| SILICONE ELASTOMER | |
| BLEND | |
| dowanol DPM | Dipropylene Glycol Methyl Ether |
| Dri-Flow Elite BN or DRY- | Aluminum Starch Octenylsuccinate (and) Boron |
| FLO Elite BN | Nitride |
| Flo-Beads SE-3207B ™ | Ethylene-methyl methacrylate copolymer |
| Dow Corning FZ-3196 | Caprylyl Methicone |
| Ganzpearl GMP-0830 ™ | Acrylates Crosspolymer |
| Granhydrogel O ™ | Water (and) Glyceryl Polyacrylate (and) 1,3- |
| | Butylene Glycol (and) PVM/MA (and) |
| | Propylparaben (and) Methylparaben |
| Granpowder Nylon ™ | Nylon-12 |
| Gransil EP-LS ™ | Polysilicone-11 (and) Laureth-12 |
| Gransurf 90 | Cetyl PEG/PPG-10/1 Dimethicone |
| Iris | C12-17 Alkanes |
| Iron Oxide Tint or Iron Oxide | Iron Oxides |
| Tint Mixture | |
| Isododecane | mixture of highly branched C12 isoparaffins, |
| | mainly the 2,2,4,6,6-pentamethylheptane isomer |
| | (typically c.a. 85%). |
| Jeechem BUGL ™ or Jeen | Butylene Glycol |
| BUGL | |
| Jeecide cap 5 | Phenoxyethanol, Caprylyl Glycol, Potassium |
| | Sorbate, Aqua, Hexylene Glycol |
| Jeensilc CPS-312 ™ | Cyclomethicone |
| Kaolin USP BC2747 | Kaolin |
| KF6013 | PEG-9 Dimethicone |
| KF-995 | CYCLOPENTASILOXANE |
| KTZ Xian Vistas ™ | Titanium Dioxide (And) Mica (And) Iron Oxide |
| | (C.I. 77491); chemical name: Mica (and) |
| | Titanium Dioxide (and) Ferrous Oxide |
| Labrafac CC ™ | Caprylic/Capric Triglyceride |
| LILAC ™ (Sonneborn) | C14-22 Alkane |
| MPDiol | Methyl Propanediol |
| Neolone PE ™ | Phenoxyethanol, Methylisothiazolinone |
| Nylon | Nylon 12 |
| Nylon 10-I2 ™ | Nylon 12 (And) Isopropyl Titanium Triisostearate |
| PC 075.3 | Hydrogen Dimethicone |
| Permethyl 99A | Isododecane |
| Pink tint mix | Iron Oxides |
| Plantacare 818 UP ™ | Coco-Glucoside; Chemical Description is “C8-16 |
| | fatty alcohol glucoside” |
| Platinum divinyl complex (for | UPAC name “1,3-Diethenyl-1,1,3,3- |
| example PT-50175F) | tetramethyldisiloxane-platinum (1:1)”; Trade |
| | name: “Platinum-divinyltetramethyldisiloxane |
| | complex”; Synonyms: Platinum(0)-1,3-divinyl- |
| | 1,1,3,3-tetramethyldisiloxane complex solution; |
| | pt(0)-1,3-divinyl-tetrame-disiloxane compl 0.100; |
| | 1,3-Divinyl-1,1,3,3-tetramethyl-disiloxane- |
| | platinum (0) |
| Pluracare ® L 64 | Poloxamer 184 (Emulsifier) |
| PMX-1184 or XIAMETER ® | Dimethicone and trisiloxane |
| PMX-1184 Silicone Fluid | |
| Polyglycol P425 | PPG-9 |
| prestige pearlescent beige | mixture of titanium and iron oxides of a beige |
| | color |
| PS123-KG | Hydrogen Dimethicone |
| RM 2051 or RM 2051 | Sodium Polyacrylate (and) Dimethicone (and) |
| Thickening Agent | Cyclopentasiloxane (and) Trideceth-6 (and) |
| | PEG/PPG 18/18 |
| Schercemol ™ 318 Ester | Isopropyl Isostearate |
| Sepiplus 400 ™ | Polyacrylate 13 (and) Polyisobutene (and) |
| | Polysorbate 20 |
| Shin Etsu KF 6038 | Lauryl PEG-9 Polymethylsiloxyethyl |
| | Dimethicone |
| Shin Etsu KSG 820 | Lauryl Dimethicone/Polyglycerin-3 |
| | Crosspolymer |
| Silsoft 034 | caprylyl methicone |
| silsoft ETS | ethyl trisiloxane |
| Simulgel EG ™ | Sodium acrylate/acryloyldimethyl taurate |
| | copolymer & Isohexadecane & Polysorbate 80 |
| SIMULGEL NS | Hydroxyethylacrylate/sodium acryloyldimethyl |
| | taurate copolymer & squalane & polysorbate 60 |
| Soft Bead B or Soft Beads B | Ethylene/Methacrylate Copolymer |
| Solagum AX | Acacia senegal gum and xanthan gum |
| SR 1000 Resin | Trimethylsiloxysilicate |
| Tint | Iron Oxides |
| TMF 1.5 | Methyl Trimethicone |
| Tween 20 | Polysorbate 20 |
| UCT-PS448.5 | Polydimethylsiloxane, Vinyldimethyl Terminated |
| Ultracolor Blue 1% dye | Water and Propylene Glycol and FD & C Blue 1 |
| USG 102 | Dimethicone/Vinyl Dimethicone Crosspolymer |
| Veegum Pro | Tromethamine Magnesium Aluminum Silicate |
| Veegum Ultra Granules | Magnesium Aluminum Silicate |
| Velvesil 125 ™ | Cyclopentasiloxane (and) |
| | C30-45 Alkyl Cetearyl Dimethicone |
| | Crosspolymer |
| Velvet Veil 310 ™ | Mica (and) Silica |
| Vitamin-A complex | retinol |
| Vitamin-C complex | ascorbic acid |
| Vitamin-E complex | Tocopherol |
| Xirona caribbean blue | Mica, Titanium Dioxide, Silica, Tin Oxide |

### Foundation Component Example 1:

Siloxane and fumed silica mixture

| | |
|---|---|
| 10,000 Cp Vinyl Dimethicone | 53% |
| 165,000 Cp Vinyl Dimethicone | 13% |
| Silica Silylate | 34% |

### Foundation Component Example 2:

Siloxane and fumed silica mixture

| | |
|---|---|
| 165,000 Cp Vinyl Dimethicone | 66% |
| Silica Silylate | 34% |

Foundation Components in Examples 1 and 2 were prepared by combining the ingredients listed above, mixing using a counter roating centrifugal mixer at 2000 RPM for 2 minutes and scraping the edges. The mixing and scraping was repeated at least twice or until the mixture became homogeneous.

### Foundation Component Example 3:

Siloxane and fumed silica preblend

| | |
|---|---|
| 10,000 Cp Vinyl Dimethicone | 8.68% |
| 165,000 Cp Vinyl Dimethicone | 2.05% |
| Silica Silylate | 6.5% |
| Dimethicone Crosspolymer | 10.35% |
| Cyclopentasiloxane | 72.42% |

Foundation Component in Example 3 was prepared by combining the ingredients listed above, mixing using a propeller mixer for 30 minutes until the mixture became homogeneous. After mixing, the sample was milled for three passes using a triple roller mill.

### Coloring Component Example 1:

Oxide Dispersion concentrations in PMX 1184

| | |
|---|---|
| Titanium Oxide SI | 69.99% |
| Yellow Iron Oxide SI | 64.029% |
| Red Iron Oxide SI | 78.894% |
| Black Iron Oxide SI | 62.363% |

### Coloring Component Example 2:

Oxide Dispersion concentrations in PMX 1184

| | |
|---|---|
| Titanium Oxide SI | 70% |
| Yellow Iron Oxide SI | 64.35% |
| Red Iron Oxide SI | 75% |
| Black Iron Oxide SI | 63% |

Coloring Components in Examples 1 and 2 were prepared by combining the ingredients listed above, mixing using a conuter roating centrifugal mixer at 1000 RPM for 2 minutes and scraping the edges. The mixing and scraping was repeated at least twice or until the mixture became homogeneous.

### Coloring Component Example 3:

Oxide Dispersion concentrations in PMX 1184

| | |
|---|---|
| Titanium Oxide SI | 72.353% |
| Yellow Iron Oxide SI | 64% |
| Red Iron Oxide SI | 75% |
| Black Iron Oxide SI | 62.357% |

Coloring Component in Example 3 was prepared by combining the ingredients listed above, mixing using a propeller mixer at 1000 RPM for 10 minutes and scraping the edges. The mixing and scraping was extended as needed to ensure a homogeneous sample and all oxides are wetted. After mixing the sample was milled for three passes using a triple roller mill.

### Coloring Component Example 4:

Monochromatic pigment concentrations of 4 shades span the range of human skin shade from light to dark and provide natural coverage

4 Base Shades

| | Titanium | Yellow Iron | Red Iron | Black Iron |
|---|---|---|---|---|
| | Oxide SI | Oxide SI | Oxide SI | Oxide SI |
| Shade | (72.353%) | (64.00%) | (75.00%) | (62.357%) |
| Light | 2.612% | 0.818% | 0.090% | 0.080% |
| Light/Med | 2.299% | 1.025% | 0.169% | 0.108% |
| Medium | 1.997% | 1.261% | 0.212% | 0.130% |
| Deep | 1.215% | 1.597% | 0.434% | 0.354% |

Per 100 g of CCP

Shading of color correcting base is achieved by adding each individual pigment while mixing to the completed base. The formula was mixed with low shear propeller mixing (500 rpm) until uniform and no color streaks are observed.

### Coloring Component Example 5:

Monochromatic pigment concentrations of 5 shades span the range of human skin shade from light to dark and provide natural coverage

5 Base Shades

| | Titanium | Yellow Iron | Red Iron | Black Iron |
|---|---|---|---|---|
| | Oxide SI | Oxide SI | Oxide SI | Oxide SI |
| Shade | (69.99%) | (64.029%) | (78.894%) | (62.363%) |
| Fair | 1.608 | 0.2 | 0.02 | 0.015 |
| Light | 1.25 | 0.38 | 0.04 | 0.035 |
| Light/Med | 1.067 | 0.46 | 0.065 | 0.042 |
| Medium | 0.883 | 0.5395 | 0.089 | 0.051 |
| Deep | 0.557 | 0.6995 | 0.176 | 0.1493 |

Per 100 g of CCP

Coloring Components in Example 5 were prepared by combining the ingredients listed above, mixing using a conuter roating centrifugal mixer at 1000 RPM for 2 minutes and scraping the edges. The mixing and scraping was repeated at least twice or until the mixture became homogeneous.

### Coloring Component Example 6:

Monochromatic pigment concentrations of 15 shades span the range of human skin shade from light to dark and provide natural coverage

15 Base Shades

| Shade number | | Titanium | Yellow Iron | Red Iron | Black Iron | |
|---|---|---|---|---|---|---|
| and under- | | Oxide SI | Oxide SI | Oxide SI | Oxide SI | |
| tone code | Ref | (69.99%) | (64.029%) | (78.894%) | (62.363%) | Base |
| 1Y | 81-65-9 | 3.136% | 0.450% | 0.060% | 0.090% | 96.264% |
| 2Y | 81-70-3 | 3.000% | 0.700% | 0.100% | 0.070% | 96.130% |
| 3Y | 81-65-10 | 3.136% | 0.476% | 0.076% | 0.100% | 96.212% |
| 4Y | 81-68-3 | 1.759% | 1.399% | 0.160% | 0.190% | 96.491% |
| 5Y | 81-69-5 | 0.999% | 2.499% | 0.214% | 0.180% | 96.108% |
| 6Y | 81-68-6 | 2.026% | 2.135% | 0.514% | 0.198% | 95.127% |
| 7Y | 81-69-8 | 2.560% | 2.400% | 0.700% | 0.500% | 93.840% |
| 8Y | 81-68-4 | 0.576% | 1.400% | 0.540% | 0.600% | 96.884% |
| 1N | 81-65-5 | 3.234% | 0.400% | 0.040% | 0.020% | 96.306% |
| 2N | 81-65-7 | 3.108% | 0.490% | 0.060% | 0.036% | 96.306% |
| 3N | 81-66-2 | 3.098% | 0.590% | 0.078% | 0.096% | 96.139% |
| 1P | 81-70-7 | 3.220% | 0.400% | 0.080% | 0.040% | 96.260% |
| 2P | 81-70-8 | 3.128% | 0.490% | 0.060% | 0.040% | 96.282% |
| 3P | 81-70-11 | 2.900% | 0.560% | 0.100% | 0.060% | 96.380% |
| 4P/E | 81-72-1 | 1.660% | 1.120% | 0.200% | 0.180% | 96.840% |

Y = yellow undertone, N = neutral, P = pink

Coloring Components in Example 6 were prepared by combining the ingredients listed above, mixing using a conuter roating centrifugal mixer at 1000 RPM for 2 minutes and scraping the edges. The mixing and scraping was repeated at least twice or until the mixture became homogeneous.

### Sun Protection Component Examples 1-8:

SPF Cocktail Compositions

| | Formula No. | | | | | | | |
|---|---|---|---|---|---|---|---|---|
| Active | 79-205 | 79-202 | 79-201 | 79-114-3 | 79-114-2 | 79-114-1 | 81-48-1 | 79-127 |
| ZnO Micro | 15.70% | 15.50% | 15.00% | 25.00% | 15.00% | 15.00% | 7.50% | 10.00% |
| (68.29% in D5) | | | | | | | | |
| TiO2 Micro | | | | | | | 2.50% | |
| Octinoxate | 7.00% | 7.00% | 7.00% | | 5.00% | 5.00% | | 4.00% |
| Avobenzone | | | | | | | | |
| Octocrylene | | | | | | | | |
| Solastay S1 | | | | | 4.00% | | | |
| In-vivo SPF | 17.6 | 14.9 | 17.6 | 14.3 | 14.9 | 13.4 | 12.2 | 13.2 |

### Sun Protection Component Examples 8-12:

SPF Cocktail Compositions

Formula No.

| | 79- | 79- | 79- | 79- |
|---|---|---|---|---|
| Active | 114-4 | 124-2 | 124-2 | 124-1 |
| ZnO Micro | | | | |
| (68.29% in D5) | | | | |
| TiO2 Micro | | | | |
| Octinoxate | 7.70% | | | |
| Avobenzone | 2.30% | 3.50% | 2.30% | 1.15% |
| Octocrylene | | 11.60% | 7.70% | 3.85% |
| Solastay S1 | 4.00% | | | |
| In-vivo SPF | 14.2 | 14.6 | 15.9 | 13.9 |

Sun protection components in Examples 8-12 were prepared by mixing the ingredients listed above with a blade at 500 RPM until the mixture was homogeneous.

### Color Corrective Composition 1

### Formulation 79-202

| Component | | Percent of |
|---|---|---|
| No. | Component | Formulation (%) |
| 1 | AN-109-PB (Foundation compound | 61.50 |
| | Sample N1) | |
| 2 | Escalol 557 | 7 |
| 3 | Dow Corning 5329 | 0.2 |
| 4 | Xiameter PMX-1184 | 0.5 |
| 5 | Nylon 12 and Isopropyl Titanium | 4.5 |
| | Triisostearate | |
| 6 | Zano 10 Plus and KF-995 | 22.7 |
| 7 | Coloring Component and KF-995 | 3.6 |

### Procedure:

Component No 1 is prepared according to the procedure of Foundation component sample N1. Components 2 to 6 are added to 1 and mixed using a propeller mixer for 30 minutes or until homogeneous. Individual iron oxide blends (e.g., as described in Coloring Component Examples 3 and 4) are added with mixing after each addition until uniform and no color streaks are observed

### Color Corrective Composition 1

### Formulation 79-202

| Component | | Percent of |
|---|---|---|
| No. | Component | Formulation (%) |
| 1 | Pigment Dispersion Mixture (Atlas | 3.6 |
| | White SI, Yellow Red Iron Oxide SI, | |
| | Black Iron Oxide SI and PMX-1184) | |
| 2 | Octinoxate | 7 |
| 3 | Nylon 12 and Isopropyl Titanium | 4.5 |
| | Triisostearate | |
| 4 | Andisil VS 10,000 | 5.3 |
| 5 | Andisl VS 165,000 | 1.3 |
| 6 | Aerosil R8200 | 3.4 |
| 7 | Dow Corning 5329 | 0.2 |
| 8 | PMX-1184 | 0.5 |
| 9 | Zano 10 Plus and KF-995 | 22.7 |
| 10 | Dow Corning 9045 | 51.5 |

### Procedure:

Component 1 was prepared by dispersing individual oxides in PMX-1184, milling the dispersion and combining the individual pigment dispersion into a Pigment Mix according to (example above). Components 4 to 6 are mixed with a dual asymmetric centrifugal mixer at 2500 RPM for 6 minutes until particulates are no longer visible (Mixture B). Pigment Mix, Mixture 2 and components 2, 3, 7 and 8 are added together and mixed with a 4-blade propeller at 800 rpm until homogenous. Component 9 is added and mixed until homogenous and finally, Component 10 is added and mixed until homogenous.

### Example 2

### Formulations: Reactive Reinforcing Component and Crosslinking Component

Examples of formulations have been described in PCT/US2011/050003, published as WO2012/030984, the teachings of which are incorporated herein by reference. Additional formulations are listed below.

### LPS026 (88-116)

| phase | ingredient | INCI | lot number | Manufacturer | % |
|---|---|---|---|---|---|
| A | DI water | | NA | | 50.0% |
| A | Jeechem | Butylene glycol | J9816G07890 | JEEN | 5.0% |
| | BUGL | | | | |
| A | Cremaphor | PEG-35 Castor Oil | 9288465680 | BASF | 5.0% |
| | EL | | | | |
| A | schercelmol | Isopropyl Isostearate | 100979775 | Lubrizol | 2.0% |
| | 318 | | | | |
| A | Jeecide | Phenoxyethanol, | J6916E0207 | JEEN | 0.5% |
| | CAP-5 | Caprylyl Glycol, | | | |
| | | Potassium Sorbate, | | | |
| | | Aqua, Hexylene Glycol | | | |
| B | PMX1184 | dimethicone and | 6462279 | Dow Corning | 37.5% |
| | | trisiloxane | | | |

Mix the components of phase A in the mixing vessel and stir until it appears uniform.
Mix the components of phase B in a separate vessel until it appears uniform.
Add phase B to Phase A (step 2 to step 1) slowly and mix until uniform.

### Formulation LPS-033 (STEP 1)

| Component | | Percent of |
|---|---|---|
| No. | Component | Formulation (%) |
| 1 | Andsil VS10,000 | 14.13 |
| 2 | Andsil VS165,000 | 3.30 |
| 3 | Andsil XL-11 | 6.92 |
| 4 | Aerosil R8200 | 8.98 |
| 5 | PMX-1184 | 13.33 |
| 6 | RM 2051 | 5.00 |
| 7 | Water | 48.01 |
| 8 | Jeecide CAP-5 | 0.33 |

### Procedure:

Components 1-4 are mixed in a beaker until the mixture is uniform and free of white particulates. Subsequently, Component 5 is added and the mixture is confirmed homogenous. Component 6 is then added to this and mixed until uniform (Mixture A). In a separate vessel, Components 7 and 8 are hand mixed until homogenous (Mixture B). Mixture B is very slowly added to Mixture A while maintaining a relatively low mixing speed. After all of Mixture B is added, the whole mixture is mixed for 10 minutes to assure homogeneity.

### Formulation 100-47-300D (STEP 11)

| Component | | Percent of |
|---|---|---|
| No. | Component | Formulation (%) |
| 1 | Andsil VS 10,000 | 14.13 |
| 2 | Andsil VS | 3.30 |
| | 165,000 | |
| 3 | Andsil XL-11 | 6.92 |
| 4 | Aerosil R8200 | 8.98 |
| 5 | PMX-1184 | 13.33 |
| 6 | RM 2051 | 3.33 |
| 7 | Covacryl MV60 | 0.43 |
| 8 | Water | 49.25 |
| 9 | Jeecide CAP-5 | 0.33 |

### Procedure:

Components 1-4 are premixed until confirmed as free of white particulates. Component 5 is added and mixed at 200 rpm until uniform (Mixture A). Components 6 and 7 are added sequentially with mixing at 500 rpm after each addition until homogeneous. (Mixture A) In a separate vessel, components 8 and 9 are hand mixed until homogenous (Mixture B). Mixture B is added to Mixture A under strong agitation, provided by a 4-blade, 40 mm propeller at 800 rpm. After all of Mixture B is incorporated, the total mixture is mixed for 5 minutes. The mixture was confirmed as homogenous.

### Formulation LPS-034 (STEP 2)

| Component | | Percent of |
|---|---|---|
| No. | Component | Formulation (%) |
| 1 | Dow 9011 Elastomer | 10.00 |
| | Blend | |
| 2 | Dow 9045 Elastomer | 10.00 |
| | Blend | |
| 3 | KF-995 | 10.00 |
| 4 | PT-50175F | 1.00 |
| 5 | Water | 28.5 |
| 6 | Plantacare 818 UP | 0.50 |
| 7 | Propylene Glycol | 20.00 |
| 8 | Glycerin USP | 4.00 |
| 9 | Jeechem BUGL | 10.00 |
| 10 | Sodium Chloride | 1.00 |
| 11 | Nylon 10-I2 | 4.5 |
| 12 | Jeecide CAP-5 | 0.5 |

### Procedure:

Components 5 and 10 are mixed until mixture is uniform. Component 12 is added to the mixture and mixed well. In a separate vessel, components 6 to 9 are mixed until the mixture is homogeneous. The mixture of components 5, 10 and 12 are then added to this mixture and stirred (Mixture A). Components 1, 2 and 3 are mixed in a separate vessel until they appear homogenous. 25% of Mixture A is slowly added to the vessel containing components 1 to 3 and mixed until the emulsion forms. Then, the rest of Mixture A is slowly added while continuously mixing. Once the mixture appears homogenous, Component 11 is slowly added and mixed until the mixture appears uniform. Finally, Component 4 is added and the mixture is stirred for a few minutes.

### Formulation 92-059-10 (REMOVER)

| Component | | Percent of |
|---|---|---|
| No. | Component | Formulation (%) |
| 1 | Water | 52.94 |
| 2 | PMX-1184 | 29.96 |
| 3 | Permethyl 99A | 9.99 |
| 4 | Jeechem BUGL | 4.24 |
| 5 | Potassium | 0.90 |
| | Phosphate Dibasic | |
| 6 | Sodium Chloride | 0.75 |
| 7 | Jeecide CAP-5 | 0.50 |
| 8 | Disodium EDTA | 0.50 |
| 9 | Maltodextrin | 0.30 |
| 10 | Ultracolor Blue | 0.09 |
| | 1% Dye | |
| 11 | Pluracare ® L 64 | 0.03 |

### Procedure:

All the components except components 2 and 3 are mixed until the mixture is uniform. Components 2 and 3 are added to the mixture to form a two-phase mixture.

### Example 3

### Evaluation of Cosmetic Benefit of Color Corrective Composition Using Image Analyses Measures

### Subjects:

Female subjects 35-65 years old with visible signs of aging in the undereye area participated in the study.

### Test Material Composition:

Color Corrective Composition 1 (formulation 79-202, see Example 1):

| Ingredient | INCI Name | Percentage |
|---|---|---|
| Pigment Mix in | Titainum Dioxide and Iron Oxides and | 3.60% |
| PMX | Trisiloxane and Dimethicone | |
| Escalol 557 | Octinoxate | 7.00% |
| Nylon 10-I2 | Nylon 12 and Isopropyl Titanium | 4.50% |
| | Triisostearate | |
| AN109-4 | Vinyl Dimethicone and Hydrogen | 10.00% |
| | Dimethicone and Silica Silylate | |
| DC 5329 | PEG-12 Dimethicone | 0.20% |
| PMX-1184 | Vinyl Dimethicone and Hydrogen | 0.50% |
| | Dimethicone and Silica Silylate | |
| 100-10 ZnO in | Zinc Oxide and Triethoxycaprylylsilane and | 22.70% |
| D5 | Cyclopentasiloxane | |
| DC 9045 | Cyclomethicone and Dimethicone | 51.50% |
| | Crosspolymer | |

### Reactive Reinforcing Component:

### Formulation 100-53 (300D)

| Component | | Percent of |
|---|---|---|
| No. | Component | Formulation (%) |
| 1 | VS 10,000 | 14.13 |
| 2 | VS 165,000 | 3.30 |
| 3 | XL-11 | 6.92 |
| 4 | Aerosil R8200 | 8.98 |
| 5 | PMX-1184 | 13.33 |
| 6 | RM 2051 | 3.33 |
| 7 | Covacryl MV60 | 0.43 |
| 8 | Water | 49.25 |
| 9 | Jeecide CAP-5 | 0.33 |

### Procedure:

Components 1-4 are premixed until confirm as free of white particulates. Component 5 is added and mixed at 200 rpm until uniform (Mixture A). Components 6 and 7 are added sequentially with mixing at 500 rpm after each addition until homogeneous. (Mixture A) In a separate vessel, components 8 and 9 are hand mixed until homogenous (Mixture B). Mixture B to was added Mixture A under strong agitation, provided by a 4-blade, 40 mm propeller at 800 rpm. After all of Mixture B is incorporated mix for 5 minutes. The mixture is confirmed as homogenous.

### Crosslinking Component:

### Formulation 100-52A (O.P)

| Component | | Percent of |
|---|---|---|
| No. | Component | Formulation (%) |
| 1 | Dow 9011 Elastomer | 10.00 |
| | Blend | |
| 2 | Dow 9045 Elastomer | 10.00 |
| | Blend | |
| 3 | KF-995 | 10.00 |
| 4 | PT-50175F | 1.00 |
| 5 | Water | 28.5 |
| 6 | Plantacare 818 UP | 0.50 |
| 7 | Propylene Glycol | 20.00 |
| 8 | Glycerin USP | 4.00 |
| 9 | Jeechem BUGL | 10.00 |
| 10 | Sodium Chloride | 1.00 |
| 11 | Nylon 10-I2 | 4.5 |
| 12 | Jeecide CAP-5 | 0.5 |

### Study Procedure:

Components 5 and 10 are mixed until mixture is uniform. Component 12 is added to the mixture and mixed well. In a separate vessel, components 6 to 9 are mixed until the mixture is homogeneous. The mixture of components 5, 10 and 12 are then added to this mixture and stirred (Mixture A). Components 1, 2 and 3 are mixed in a separate vessel until they appear homogenous. 25% of Mixture A is slowly added to the vessel containing components 1 to 3 and mixed until the emulsion forms. Then, the rest of Mixture A is slowly added while continuously mixing. Once the mixture appears homogenous, Component 11 is slowly added and mixed until the mixture appears uniform. Finally, Component 4 is added and the mixture is stirred for a few minutes.

### Procedure:

The panelist was asked to remove her under-eye area makeup and moisture using a CVS moist towlette. Then, the same area was cleaned using a water-wetted kimwipe. Then the following procedure was used to apply the products under each eye:

0.04 g of the medium colored color-corrective composition was applied to the under-eye area using a dabbing motion. After a thin and uniform layer of color corrective composition was applied, 0.06 g of reactive reinforcing component (step 1) was applied. This applied by evenly distributing the treatment in the under-eye area using 3 dabs. Then from the inner corner of the eye, the treatment was spread to the crow's feet area. Using a single direction of application, the treatment was spread uniformly via 6-8 strokes. Then 0.09 g of crosslinking component was applied on top of the treatment area. Using the same dabbing, then spreading method for the treatment, the crosslinking component is gently placed on top without mixing or disturbing the bottom 2 layers. When finished, the edges of the application area were gently smoothed out via a small wiping motion.

### Photo Set Up Capture:

To ensure maximum repeatability in panelist placement for every photograph taken for product performance evaluation, a Head Positioning System (HPS) was created. This HPS had two configurations: forehead-only evaluations and a whole face evaluation. In both configurations, a model 819 series table clamped chin-rest from Applied Science Laboratories (ASL) was used as a base to mount the two different configurations to a table. Two cameras were used to capture the subject from two different angles. The first camera (normal shot) was positioned face-on such that line of the lens through the camera was positioned relative to the plane of the subject's face at an angle of approximately 90°. A second camera (45° shot) was positioned to the subject's left such that the line of the lens through the camera was positioned relative to the plane of the subject's face at an angle of approximately 45° capturing primarily the left side of the subject's face. The position of the cameras relative to the chin-rest was kept fixed.

In the first configuration, an ASL cheek-rest (819-2155) was mounted to the ASL chin-rest. In this setup, the panelist's head was positioned such that the line formed from the center of the camera lens to the area of evaluation is normal to the area of evaluation on the forehead. In the second configuration, an ASL forehead-rest (819-2150) was attached to the chin-rest. In this setup, the chin-rest cup was positioned such that the horizontal bar on the forehead-rest was situated at the panelist's horizontal hairline, maximizing the area of evaluation in every photograph.

Lighting for the photography consisted of two Calumet Quattro fluorescent lamps (CF0003) with four Calumet 35 Watt 5500K daylight color temperature fluorescent lights (OL2003) placed in front of and on either side of the panelist, angled to point directly at the panelist. A glare stop polarizing filter from Visual Pursuits, Inc. was also placed on the front of each lamp. The lights were allowed to warm up for at least 10 minutes prior to taking any photographs. In addition to the lighting, a circular polarizing filter was used on each of the camera lenses to control the type of light in each photograph.

For each evaluation, two sets of pictures were taken for the normal shot camera. In the first set, the camera's circular polarizing filter was configured such that its polarization was parallel to the polarization of the fluorescent lights, giving a picture that highlighted the shine as well as the fine wrinkles, pores and skin texture. In the second set, the circular polarizing filter was configured such that the polarization was perpendicular (or cross) to the polarization of the fluorescent lights yielding a result that eliminated all glare and shows the underlying skin tone, discoloration, and deep wrinkles.

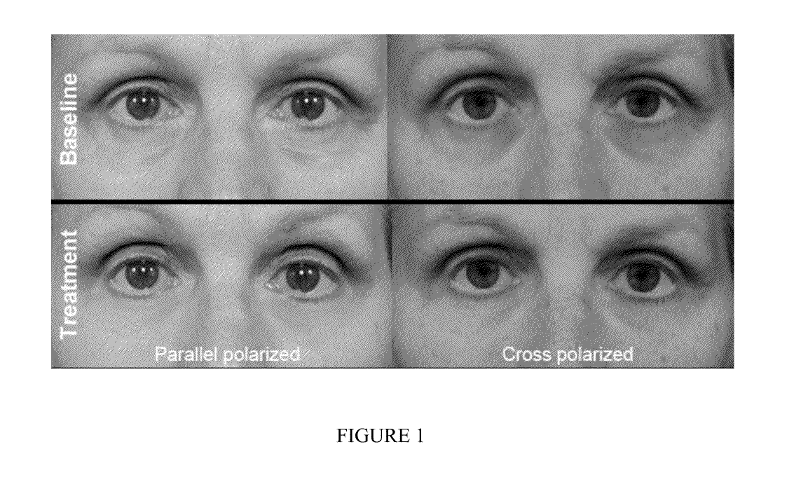

FIG. 1 shows the photographs of two subjects taken before and after application of the color corrective composition in conjunction with a film forming agent.

### Example 4

### Sun Protective Properties of Color Corrective Composition

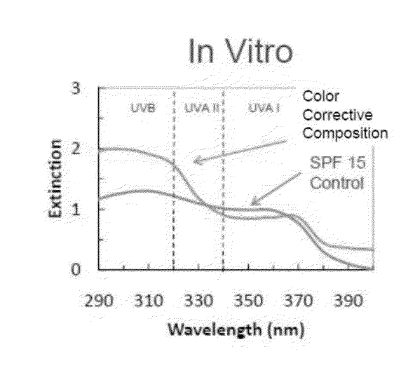

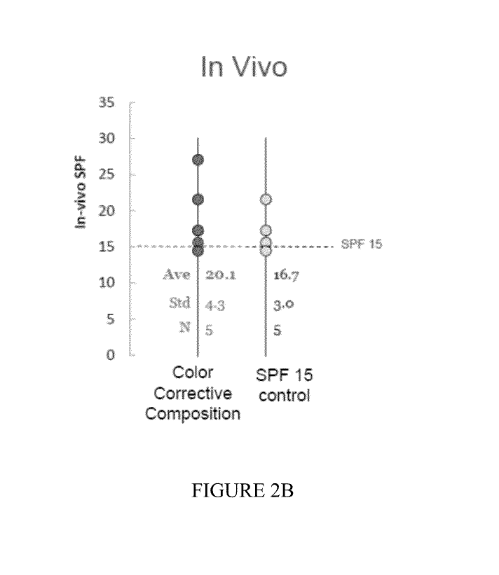

The sun protective properties of Color Corrective Composition 1 (formulation 79-202, see Example 1) were investigated. In vitro testing was conducted using the protocol described in the Jun. 17, 2011 FDA final rule on critical wavelength calculation. In vivo testing was conducted using the protocol described in the 2011 FDA final monograph, following CFR Parts 210 and 310. FIG. 2A is a graph showing the improved in vitro UVB protection of an exemplary composition. FIG. 2B is a graph showing the improved in vivo UVB protection achieved by the color corrective composition.

### Example 5

### Evaluation of Longevity and Stability of the Color Corrective Composition

In order to capture the longevity of the color correcting composition, a panelist was asked to wear commercial make-up, which includes Lancome Foundation, Shu Emura Bronzer, and Laura Mercier Powder, on half side of her face and the color corrective composition (79-202) with the reactive reinforcing component (100-69 (300D); equivalent to 100-53 (300D), as described in Example 3) and crosslinking component (100-55 (O.P), equivalent to 100-52A, as described in Example 3) on the other side of her face. The panelist was photographed at baseline, 3 hours post product application, and after splashing her face with water for 10 seconds. After wetting her face, the panelist gently dabbed a kimwipe all over face to dry the skin surface.

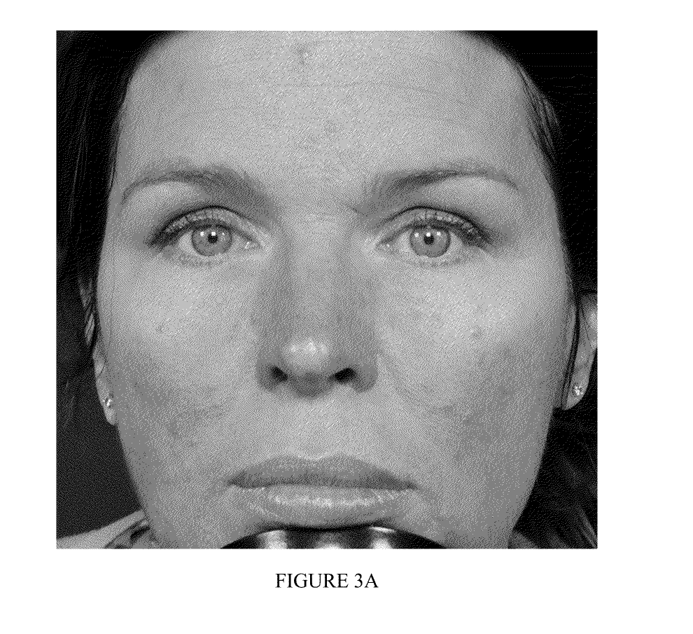

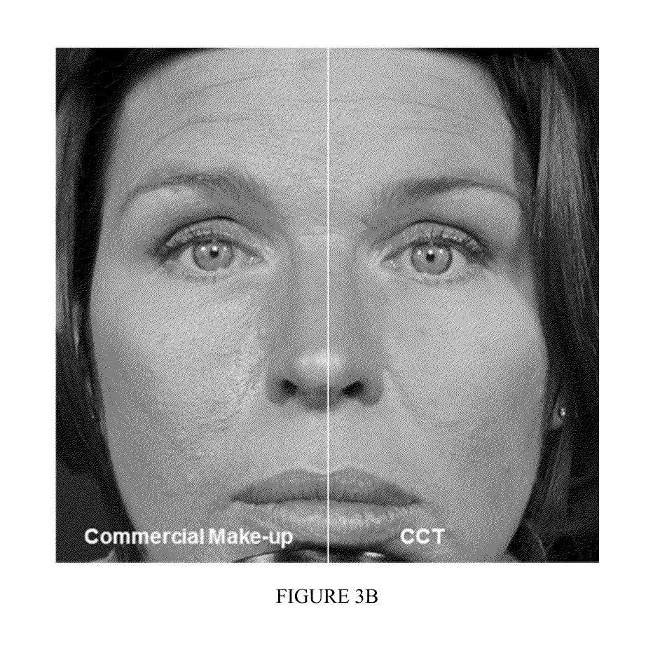

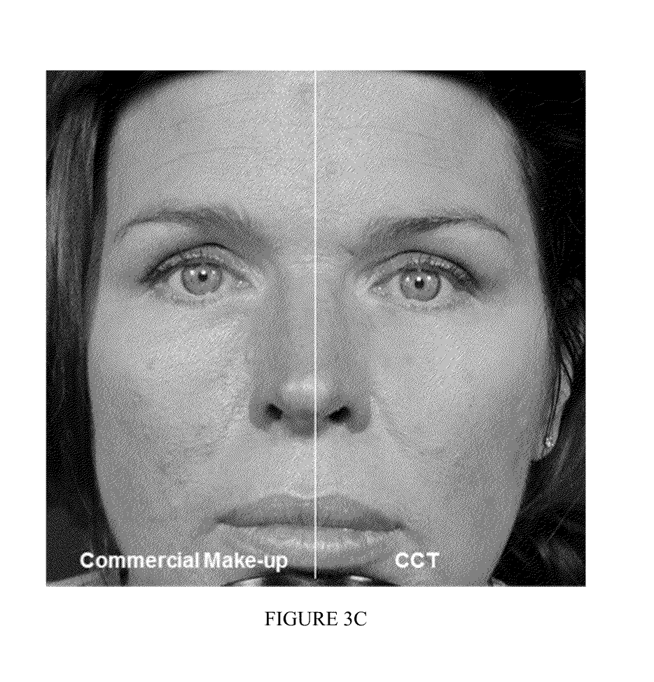

As seen from FIG. 3A, the color corrective composition (labeled as “CCT” in FIGS. 3A-3C) is able to provide the same level of coverage and aesthetics as commercial make-up. However, it performances significantly higher with shine prevention and color longevity when challenged with other external factors (FIG. 3B). When the baseline picture is compared with the post water splashing picture, it is evident that on the commercial make-up side, the panelist's redness is more prominent than the side with the color corrective composition (FIG. 3C).

In addition to expert evaluation of photographs or live users, long lasting color correction achieved with the color corrective composition can be measured by a chroma meter (http://www.konicaminolta.com/instruments/products/color-measurernent/colorimeters/cr400-410/index.html) or quantitative digital photography analysis methods http://onlinelibrary.wiley.com/doi/10.1111/j.1600-0846.2010.00468.x/abstract, the teachings of which are all incorporated herein by reference. The meter can quantify the color shift on a live subject before and after the product has been applied.

The same instrument can be used to evaluation the stability of the color within the color corrective composition by measuring the color variability across the formulation. The equivalence of chroma measurement from the top and the bottom of the formulation can be used to confirm color homogeneity and formula stability.

### Example 6

### Evaluation of Longevity and Stability of the Color Corrective Composition

The longevity of the color can be determined by comparing the color correcting function of the color corrective composition to that of conventional make up. Conventional make up loses significant potency within 4-8 hrs of application, however the color corrective composition provides greater then 12 hrs of coverage. The color correcting performance at 12 hrs can be measured by quantitative color analysis (chroma meter or quantitative photography) or via expert human assessment of color corrected skin.

In addition to normal long term wear, the longevity can be assessed under stress conditions such as exposure to water (associated with rain or sweating) or mechanical abrasion (associated with touching or rubbing by clothes). The assessment can be made on live subjects or on artificial tightly controlled surfaces. The following representative protocol can be used: Water Protocol for in-vitro spf testing

The color corrective composition is compared as a solo ingredient (not under a film) to commercial foundation I.e.: Lancôme, for example, by the method from Sun Care Lab, which gives 0.075 g for 96 well plate surface.

1. Cover a well plate with Saran wrap.
2. Spread test spf product evenly on the surface.
3. UV spectrophotometer—define protocol—290 and 400 wavelength.
4. Measure the absorbance
5. Submerse the entire plate in water for one hour
6. Gently dab off excess water using a dry towel.
7. Re-measure the absorbance (Step 3-4)
8. Compare the decrease in absorbance between the T-0 Measurement.

### EQUIVALENTS

Those skilled in the art will recognize, or be able to ascertain using no more than routine experimentation, numerous equivalents to the specific polypeptides, nucleic acids, methods, assays and reagents described herein. Such equivalents are considered to be within the scope of this invention and are covered by the following claims.

What is claimed is:

1. A method of enhancing or providing a benefit to skin comprising the steps of:
a) applying a benefit-inducing layer to the skin;
b) applying a reactive reinforcing component to said benefit-inducing layer; and
c) applying a reactive reinforcing component to said reactive reinforcing component, wherein said cross-linking component facilitates in situ cross-linking of the reactive reinforcing component and the benefit-inducing layer, thereby enhancing or providing a benefit to skin.

2. A method of changing the color of skin comprising the steps of:
a) applying a color corrective composition to the skin;
b) applying a reactive reinforcing component to color corrective composition; and
c) applying a cross-linking component to said reactive reinforcing component, wherein said cross-linking component facilitates in situ cross-linking of the reactive reinforcing component and the color corrective composition, thereby changing the color of the skin.

3. The method of claim 2, wherein the color corrective composition comprises:
a) a foundation component;
b) a coloring component; and
c) a stabilizer component, wherein the foundation and stabilizer components stabilize the coloring component in the composition.

4. The method of claim 3, wherein the foundation component comprises vinyl dimethicone and silica silylate.

5. The method of claim 4, wherein the vinyl dimethicone is comprised of a low viscosity vinyl dimethicone.

6. The method of claim 4, wherein the vinyl dimethicone is comprised of a high viscosity vinyl dimethicone.

7. The method of claim 4, wherein the vinyl dimethicone is comprised of a low viscosity vinyl dimethicone and a high viscosity vinyl dimethicone.

8. (canceled)

9. (canceled)

10. The method of claim 1, wherein the foundation component comprises vinyl dimethicone and silica silylate in a ratio of about 2:1.

11. The method of claim 1, wherein the foundation component comprises 1 to 30% of the composition.

12. (canceled)

13. The method of claim 3, wherein the coloring component comprises an inorganic oxide pigment.

14. (canceled)

15. (canceled)

16. The method of claim 13, wherein the pigment is dispersed in a pigment solubilizer.

17. (canceled)

18. (canceled)

19. (canceled)

20. The method of claim 16, wherein the pigment and pigment solubilizer comprise 0.5% to 8% of the composition.

21. (canceled)

22. The method of claim 13, wherein the coloring component further comprises a pigment stabilizer.

23. The method of claim 22, wherein the pigment stabilizer is selected from an alkyl dimethicone, an alkyl ester dimethicone, an alkyl ether dimethicone or a combination thereof.

24. (canceled)

25. (canceled)

26. The method of claim 13, wherein the pigment stabilizer comprises 0.2% to 0.5% of the composition.

27. (canceled)

28. The method of claim 3, wherein the stabilizer component comprises elastomer beads, crosspolymers, or a combination thereof.

29. (canceled)

30. (canceled)

31. The method of claim 28, wherein the stabilizer component comprises 30% to 70% of the composition.

32. (canceled)

33. The method of claim 1, wherein the composition further comprises a sun protection component, a sensory enhancing component or a combination thereof.

34. The method of claim 33, wherein the composition further comprises a sun protection component.

35. The method of claim 34, wherein the sun protection component comprises a sunscreen.

36.-43. (canceled)

44. The method of claim 2, wherein the composition further comprises a sensory component.

45. The method of claim 44, wherein the sensory enhancing component comprises elastomer beads, inorganic powders, organic beads or a combination thereof.

46. (canceled)

47. (canceled)

48. The method of claim 44, wherein the sensory enhancing component comprises 0% to 10% of the composition.

49. The method of claim 44, wherein the sensory enhancing comprises 4% to 5% of the composition.

50. The method of claim 2, wherein the color corrective composition further comprises one or more therapeutic agents.

51. (canceled)

52. (canceled)

53. A color corrective composition comprising:
a. a foundation component;
b. a coloring component; and
c. a stabilizer component, wherein the foundation and stabilizer components stabilize the coloring component in the composition and enhance the longevity of the color when coated with a film.

54. A color corrective composition comprising:
a. a foundation component;
b. a coloring component;
c. a stabilizer component, and
d. a sun protection component, wherein the foundation and stabilizer components enhance the sun protection when coated with a film.

55. The color corrective composition of claim 53, wherein the color corrective composition further comprises one or more therapeutic agents.

56. (canceled)

57. A method of changing the color of skin comprising the steps of applying to skin a composition comprising the color corrective composition of claim 53, thereby changing the color of the skin.

58. (canceled)

59. The color corrective composition of claim 54, wherein the color corrective composition further comprises one or more therapeutic agents.

60. A method of changing the color of skin comprising the steps of applying to skin a composition comprising the color corrective composition of claim 54, thereby changing the color of the skin.
